(19) United States
(12) Patent Application Publication
(10) Pub. No.: US 2023/0168256 A1
(43) Pub. Date: Jun. 1, 2023

(54) METHODS FOR USING PROTEIN BIOMARKERS IN IDIOPATHIC PULMONARY FIBROSIS

(71) Applicant: Board of Regents, The University of Texas System, Austin, TX (US)

(72) Inventors: Nagarjun Konduru Venkata, Tyler, TX (US); Sitaramaraju Adduri, Tyler, TX (US)

(73) Assignee: Board of Regents, The University of Texas System, Austin, TX (US)

(21) Appl. No.: 17/894,833

(22) Filed: Aug. 24, 2022

Related U.S. Application Data
(60) Provisional application No. 63/236,854, filed on Aug. 25, 2021.

(51) Int. Cl. G01N 33/68; G01N 33/574

(52) U.S. Cl. CPC G01N 33/6893; G01N 33/57484; G01N 2800/52; C12Q 2600/158; G01N 2800/12; G01N 2800/50

(57) ABSTRACT

The present disclosure includes exosomal protein biomarkers for differential diagnosis of idiopathic pulmonary fibrosis including a five-protein signature determined using mass spectrometry-based proteiomic analysis of plasma extracellular vesicles (EVs) for differential diagnosis of idiopathic pulmonary fibrosis.

### CROSS-REFERENCE TO RELATED APPLICATION

This application claims the benefit of U.S. Provisional Application No. 63/236,854, filed Aug. 25, 2021, which is incorporated herein by reference in its entirety.

### FIELD OF THE INVENTION

The present disclosure includes exosomal protein biomarkers for measuring levels of novel biomarkers for idiopathic pulmonary fibrosis (IPF).

### STATEMENT REGARDING PRIOR DISCLOSURES BY THE INVENTOR OR A JOINT INVENTOR

Applicant designates the following article as a grace period publication in order to expedite examination of the application in accordance with 37 CFR 1.77(b)(6) and MPEP 608.01(a): “A novel protein signature from plasma extracellular vesicles for non-invasive differential diagnosis of idiopathic pulmonary fibrosis” displayed in medRxiv.org on May 14, 2021. The disclosures of the article are incorporated herein by reference in their entirety for all purposes.

### BACKGROUND OF THE INVENTION

Idiopathic pulmonary fibrosis (IPF) is a fibrosing interstitial pneumonia of unknown etiology that often leads to respiratory failure. With a median survival of 3.8 years, IPF appears to be more lethal than many types of cancer. Despite recent advances in understanding and treatment of IPF from research and clinical perspectives, there is still no cure for IPF. Advancements in research, and treatment of patients, are hampered by key challenges including the early identification of IPF in patients. (White, E. S., et al., “Challenges for Clinical Drug Development in Pulmonary Fibrosis,” Front Pharmacol. 2022: 13:823085.)

Diagnosis at early stages and therapeutic intervention before the lung function is severely impaired can improve treatment response and thereby prolong survival. However, diagnosis of IPF has remained challenging and often requires a multidisciplinary approach involving pulmonologists, radiologists and pathologists with extensive expertise in the evaluation of patients with interstitial lung diseases (ILDs). While the key features of IPF on high-resolution computed tomography (HRCT) or a multidisciplinary team consultation are important for accurate diagnosis of IPF, access to these key elements of care is a common challenge. In addition, for accurate diagnosis of IPF, surgical lung biopsy is often required to exclude the possibility of other interstitial lung diseases (ILDs). In this respect, HRCT imaging is critical for diagnostic evaluation of IPF. However, several other ILDs often exhibit radiologic patterns similar to IPF on HRCT, which makes diagnosis of the disease difficult. Therefore, it would be highly advantageous to have diagnostic methods for IPF that can be used to readily distinguish IPF from other ILDs. Even more, there is a long-felt and unmet need for a non-invasive method for distinguishing IPF from other ILDs.

A vast majority of studies investigated the diagnostic efficacy of individual proteins for differential diagnosis, which showed poor sensitivity and specificity. (See, e.g., Greene, K. E., et al., “Serum surfactant proteins-A and -D as biomarkers in idiopathic pulmonary fibrosis,” Eur. Respir. J. 2002: 19(3):439-46; Morals et al., “Serum metalloproteinases 1 and 7 in the diagnosis of idiopathic pulmonary fibrosis and other interstitial pneumonias,” Respir. Med. 2015: 109(8):1063-68; White et al., “Plasma Surfactant Protein-D, Matrix Metalloproteinase-7, and Osteopontin Index Distinguishes Idiopathic Pulmonary Fibrosis from Other Idiopathic Interstitial Pneumonias,” Am. J. Respir. Crit. Care Med. 2016: 194(10):1242-51; Rosas et al., “MMP1 and MMP7 as potential peripheral blood biomarkers in idiopathic pulmonary fibrosis,” PLoS Med. 2008: 5(4):e93.) Due to etiological and molecular complexity of IPF and other ILDs, use of a single biomarker to differentiate IPF from other closely resembling ILDs has not been successful. Notably, the current ATS/ERS/JRS/ALAT clinical practice guidelines strongly recommend against use of serum or plasma biomarkers such as MMP9, SFTPD, CCL18 and KL-6 for the purpose of distinguishing IPF from other ILDs, owing mainly to their poor efficacy. (Raghu et al., “Diagnosis of Idiopathic Pulmonary Fibrosis. An Official ATS/ERS/JRS/ALAT Clinical Practice Guideline,” Am. J. Respir. Crit. Care Med. 2018: 198(5):e44-e68.) There is an unmet need for a cohort of suitable biomarkers which distinguish IPF from other ILDs to allow for diagnosis of this difficult to detect disease.

While the majority of prior studies evaluated selected genes and/or proteins with a putative role in the pathology of the ILDs for their efficacy as diagnostic markers, their diagnostic efficacy has been unsatisfactory. There is a need for a diagnostic method or tool which can be used to differentiate IPF from other ILDs non-invasively, in resource-limited settings, and/or in situations when a definitive diagnosis cannot be made based on HRCT.

### SUMMARY OF THE INVENTION

In some aspects, the present disclosure includes a method of preparing a human subject sample containing extracellular vesicles (EVs) and a subpopulation enriched in exosomes comprising: extracting a human subject sample; producing a sample from said human subject sample, wherein the human subject sample contains EVs and exosomes; performing proteomic analysis of plasma extracellular vesicles (EVs) in the sample containing EVs and exosomes. In some aspects, the method further includes quantifying levels of the at least two of High mobility group box protein 1 (HMGB1), surfactant protein B (SFTPB), Aldolase A (ALDOA), calmodulin like 5 (CALML5) and Talin-1 (TLN1) in plasma extracellular vesicles (EVs) in the sample containing EVs and exosomes.

In some aspects, the present disclosure includes a method of using a biomarker panel to determine levels of at least two, three, four, or all of High mobility group box protein 1 (HMGB1), surfactant protein B (SFTPB), Aldolase A (ALDOA), calmodulin like 5 (CALML5) and Talin-1 (TLN1) in plasma extracellular vesicles (EVs) in a human subject sample containing EVs and exosomes comprising: extracting a human subject sample containing EVs and exosomes; performing proteomic analysis of plasma EVs in the human subject sample containing EVs and exosomes; quantifying levels of the at least two, three, four, or all of HMGB1, SFTPB, ALDOA, CALML5 and TLN1 in plasma EVs in the human subject sample.

In some aspects, the present disclosure includes a method of treating a subject having IPF comprising using a biomarker panel to determine levels of at least two, three, four, or all of High mobility group box protein 1 (HMGB1), surfactant protein B (SFTPB), Aldolase A (ALDOA), calmodulin like 5 (CALML5) and Talin-1 (TLN1) in plasma EVsin a human subject sample containing EVs and exosomes comprising: extracting a human subject sample containing EVs and exosomes; performing proteomic analysis of plasma EVs in the human subject sample containing EVs and exosomes; quantifying levels of the at least two of HMGB1, SFTPB, ALDOA, CALML5 and TLN1 in plasma EVs in the human subject sample; determining if levels of the at least two, three, four, or all of HMGB1, SFTPB, ALDOA, CALML5 and TLN1 in plasma EVs in the human subject sample are elevated compared to a pre-determined baseline level, and administering a therapeutic for IPF to the subject. In some aspects, the therapeutic is pirfenidone, nintedanib, or a combination thereof.

In some aspects, the present disclosure includes methods for diagnosing idiopathic pulmonary fibrosis (IPF) by identifying a protein signature in extracellular ventricles (EVs) that distinguishes IPF from other non-IPF interstitial lung diseases (ILDs) and healthy subjects. In one aspect, a method for diagnosing IPF uses exosomal protein biomarkers for differential diagnosis of idiopathic pulmonary fibrosis including a protein signature. In some aspects, the protein signature may be determined using mass spectrometry-based proteomic analysis of plasma EVs for differential diagnosis of IPF.

In some aspects, the present disclosure includes diagnosing idiopathic pulmonary fibrosis (IPF) using exosomal protein biomarkers, comprising: performing proteomic analysis of plasma EVsin a human subject sample containing EVs and exosomes; identifying a protein signature for at least two exosomal protein biomarkers for IPF; using the protein signature to determine if the at least two exosomal protein biomarkers for IPF are elevated compared to a predetermined baseline level.

In some aspects, the present disclosure includes screening for idiopathic pulmonary fibrosis (IPF) in a noninvasive or minimally invasive manner using exosomal protein biomarkers comprising: performing proteomic analysis of plasma EVs in a human subject sample containing EVs and exosomes; identifying a protein signature for at least two protein biomarkers selected from the group consisting of: High mobility group box protein 1 (HMGB1), surfactant protein B (SFTPB), Aldolase A (ALDOA), calmodulin like 5 (CALML5) and Talin-1 (TLN1); and using the protein signature to determine if the at least two biomarkers protein biomarkers are elevated compared to a predetermined baseline level for the at least two biomarkers. As used herein, non-invasive refers to techniques that do not require extraction of tissue. For example, use of a human blood plasma sample is referred to as a non-invasive or minimally invasive extraction.

In some aspects, the present disclosure includes a diagnostic test for idiopathic pulmonary fibrosis (IPF) comprising a protein biomarker panel comprising at least two exosomal protein biomarkers for IPF.

In some aspects, the present disclosure includes a biomarker panel comprising at least two exosomal protein biomarkers for IPF.

In some aspects, the present disclosure includes a method for diagnosing IPF using exosomal protein biomarkers comprising performing proteomic analysis of plasma EVs in a human subject sample, identifying a protein signature, and validating the protein signature. In one aspect, the exosomal protein biomarkers comprise SFTPB, ALDOA, HMGB1, CALML5, and TLN1. In some aspects, a definitive diagnosis cannot be made based on high-resolution computed tomography (HRCT). In certain aspects, the present disclosure includes implementing the method as an addition to HRCT and screening for idiopathic pulmonary fibrosis.

In certain aspects, the present disclosure includes method for diagnosing and differentiating IPF from other ILDs using exosomal protein biomarkers is provided. In one aspect, the exosomal protein biomarkers comprise SFTPB, ALDOA, HMGB1, CALML5, and TLN1. In some aspects, a definitive diagnosis cannot be made based on HRCT. In certain aspects, the present disclosure includes implementing the method as an addition to HRCT and screening for idiopathic pulmonary fibrosis.

In certain aspects, the present disclosure includes a method for screening for IPF in a noninvasive or minimally invasive manner using exosomal protein biomarkers is provided. In one aspect, the exosomal protein biomarkers comprise SFTPB, ALDOA, HMGB1, CALML5, and TLN1. In some aspects, a definitive diagnosis cannot be made based on HRCT. In certain aspects, the present disclosure includes implementing the method as an addition to HRCT and screening for idiopathic pulmonary fibrosis.

In certain aspects, the present disclosure includes a method for diagnosing IPF comprising extracting a protein signature from plasma EVs is provided.

In certain aspects, the human subject sample is not blood serum, not broncho-alveolar lavage fluid (BALF), not inflammatory cells, and/or not hyperplasic epithelial cells.

In certain aspects, the present disclosure includes a diagnostic test for IPF is provided comprising a protein biomarker panel. The protein biomarker panel can comprise exosomal protein biomarkers. In one aspect, the exosomal protein biomarkers comprise SFTPB, ALDOA, HMGB1, CALML5, and TLN1. In some aspects, a definitive diagnosis cannot be made based on HRCT. The diagnostic test can be implemented as an addition to HRCT and screening for idiopathic pulmonary fibrosis.

In certain aspects, the present disclosure includes a biomarker panel for diagnosis of IPF in a noninvasive or minimally invasive manner is provided. The biomarker panel can discriminate IPF from healthy subjects and other ILDs. The panel can comprise exosomal protein biomarkers. In one aspect, the exosomal protein biomarkers comprise SFTPB, ALDOA, HMGB1, CALML5, and TLN1. In certain aspects, the present disclosure includes implementing the biomarker panel as an addition to HRCT and screening for idiopathic pulmonary fibrosis.

### BRIEF DESCRIPTION OF THE FIGURES

The accompanying figures, which are incorporated herein and form part of the specification, illustrate various aspects of the present disclosure and, together with the description, further serve to explain the principles of the disclosure and to enable a person skilled in the pertinent art to make and use the aspects disclosed herein.

The patent or application file contains at least one drawing executed in color. Copies of this patent or patent application publication with color drawing(s) will be provided by the Office upon request and payment of the necessary fee.

FIG. 1 shows a representative cryo-electronic micrograph of EVs isolated from peripheral blood plasma of patients.

FIG. 2 shows a graph depicting size and quantification of plasma EVs measured by Nanoparticle Tracking Analysis against 0.1 μm fluorescent polystyrene beads standard.

FIG. 3 shows a western blot analysis detecting common EV markers for two representative plasma EV samples. The presence of EV membrane markers (CD9, CD81) and an internal marker (TSG101) and the absence of an endoplasmic reticulum marker (Calnexin) are shown.

FIG. 4 shows a western blot analysis demonstrating EV preparations exhibiting low and similar background levels of plasma proteins contamination across samples.

FIG. 5 shows a graph depicting a principle component analysis indicating that the protein profiles of EVs from healthy subjects, CHP, NSIP, and IPF clustered according to the conditions of the lungs.

FIG. 6 shows a flow chart depicting the study design including steps for systematic discovery of EV protein biomarker signature.

FIG. 7 shows a plot depicting cross validation error at different values of tuning parameter (λ) in lasso regression in cohort-I. The red dotted curve shows the estimated binomial deviance based on 5-fold cross-validation while the whiskers represent ±1 standard error, respectively. The vertical dotted lines show the locations of λ.min and λ.1se (lambda.min: λ of minimum mean cross-validated error, and lambda.1se: largest value of λ such that error is within 1 standard error of the cross-validated errors for lambda.min). The numbers across the top show the number of nonzero coefficient estimates. SE means standard error. λ.min is the value of λ at which cross validation error is the least and λ.1se is the largest value of λ within 1 standard error of λ.min.

FIG. 8 shows graphs depicting, as part of preliminary validation, the levels of five proteins in plasma EVs of 24 IPF and 23 other-ILDs assessed using sandwich ELISA with purified recombinant human protein as the standard. The asterisks *, *** and **** denote p<0.05, p<0.001 and p<0.0001, respectively.

FIG. 9 shows receiver operating characteristic (ROC) curves for a logistic regression model generated for five proteins on 24 IPF and 23 other-ILD patients.

FIG. 10 shows graphs depicting, as part of extended validation, expression levels of three proteins in plasma EVs of 34 IPF and 46 other-ILDs measured using sandwich ELISA with purified recombinant human protein as the standard. The asterisks *, *** and **** denote p<0.05, p<0.001 and p<0.0001, respectively.

FIG. 11 shows receiver operating characteristic (ROC) curves for a logistic regression model generated for two proteins on 34 IPF patients and 46 other-ILDs.

FIG. 12 shows a waterfall plot depicting risk scores calculated from predictive probabilities from a logistic regression model for each patient in extended validation of the three-protein signature in ascending order. The risk scores were computed by subtracting 0.5 from the predicted probabilities of each subject included in the model. Clinical and demographic data for each patient is color coded and shown at the bottom of the waterfall plot.

FIG. 13 shows a table summarizing the logistic regression model generated for different classifiers for discriminating IPF from other ILDs.

FIG. 14 shows graphs depicting, as part of preliminary validation, the levels of five proteins in plasma EVs of 24 IPF and 12 HS assessed using sandwich ELISA with purified recombinant human protein as the standard. The asterisks *, **, *** and **** denote p<0.05, p<0.01, p<0.001 and p<0.0001, respectively.

FIG. 15 shows ROC curves for a logistic regression model generated for five proteins on 24 IPF patients and 12 healthy subjects.

FIG. 16 shows graphs depicting, as part of extended validation, the levels of two proteins (CALML5 and HMGB1) in plasma EVs assessed using sandwich ELISA of 34 IPF and 24 HS with purified recombinant human protein as the standard. The asterisks *, **, *** and **** denote p<0.05, p<0.01, p<0.001 and p<0.0001, respectively.

FIG. 17 shows ROC curves for a logistic regression model generated for two proteins on 34 IPF patients and 24 healthy subjects.

FIG. 18 shows a waterfall plot depicting risk scores calculated from predictive probabilities from a logistic regression model for each patient in extended validation of the two-protein signature in ascending order. The risk scores were computed by subtracting 0.5 from the predicted probabilities of each subject included in the model. Clinical and demographic data for each patient is color coded and shown at the bottom of the waterfall plot.

FIG. 19 shows a table summarizing the logistic regression model generated for different classifiers for discriminating IPF from healthy subjects.

FIG. 20 shows a heatmap depicting the top ten up- and down-regulated proteins in a comparison between healthy subjects against all ILDs.

FIG. 21 shows a graph depicting potential pathways involved in the pathogenesis of IPF as identified by EV proteome and lung transcriptome.

### DETAILED DESCRIPTION OF THE INVENTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

The disclosed subject matter now will be described more fully hereinafter with reference to the accompanying Figures, in which some, but not all aspects of the presently disclosed subject matter are shown. The presently disclosed subject matter may be embodied in many different forms and should not be construed as limited to the aspects set forth herein; rather, these aspects are provided so that this disclosure will satisfy applicable legal requirements. Indeed, many modifications and other aspects of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific aspects disclosed and that modifications and other aspects are intended to be included within the scope of the appended claims.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

Before the present invention is described in detail below, it is to be understood that this invention is not limited to the particular methodology, protocols, and reagents described herein as these may vary. It is also to be understood that the terminology used herein is for the purpose of describing particular aspects only, and is not intended to limit the scope of the present invention which will be limited only by the appended claims. Unless defined otherwise, all technical and scientific terms used herein have the same meanings as commonly understood by one of ordinary skill in the art to which this invention belongs.

Idiopathic pulmonary fibrosis (IPF) is a chronic and progressively fibrosing interstitial pneumonia of unknown etiology that often leads to respiratory failure. (Wuyts et al., “Differential diagnosis of usual interstitial pneumonia: when is it truly idiopathic?”, Eur. Respir. Rev. 2014: 23(133):308-19.) With a median survival of 3.8 years, IPF appears to be more lethal than many cancer types. (Id.) Diagnosis at early stage and therapeutic intervention before the lung function is severely impaired could potentially improve treatment response and thereby prolong survival. (Flaherty et al., “Idiopathic interstitial pneumonia: what is the effect of a multidisciplinary approach to diagnosis?”, Am. J. Respir. Crit. Care Med. 2004: 170(8):904-10.)

High-resolution computed tomography (HRCT) is a crucial diagnostic component in clinical diagnosis of IPF. A clear morphologic pattern of usual interstitial pneumonia (UIP) on HRCT indicates IPF. However, UIP is not synonymous with IPF as other interstitial lung diseases (ILDs) including chronic hypersensitivity pneumonitis (CHP), and nonspecific interstitial pneumonia (NSIP), etc., may also exhibit a similar pattern. (Wuyts et al.) Therefore, accurate diagnosis of IPF involves exclusion of other ILDs such as CHP and NSIP which necessitates inquiry into patient's history and sometimes acquisition of surgical lung biopsy or broncho-alveolar lavage fluid.

There has been enormous interest in identifying biomarkers for discerning IPF from other ILDs and to aid in IPF diagnosis. Several previous studies have reported biomarkers for differential diagnosis of IPF. (See, e.g., Greene et al.; Ishii et al., “High serum concentrations of surfactant protein A in usual interstitial pneumonia compared with non-specific interstitial pneumonia,” Thorax 2003: 58:52-57; Morals et al.,; Onishi et al., “Clinical features of chronic summer-type hypersensitivity pneumonitis and proposition of diagnostic criteria,” Respir. Investig. 2020: 58(1):59-67; White et al.) The majority of these studies used the classical approach of biomarker identification wherein genes and/or proteins with a putative role in the pathology of IPF were evaluated for their efficacy as diagnostic markers, but their diagnostic efficacy has been unsatisfactory. Notably, different ILDs with fibrosis may share a similar molecular landscape between themselves. Therefore, high-throughput analyses may help in discovering molecular changes unique to each ILD.

High-throughput analyses have recently attracted attention for identification of disease biomarkers owing to their ability to quantify thousands of molecules in a single screening. The advantage of this approach is that 1) the biomarkers could be selected from a large pool of genes and/or proteins and not limited to those with an evidence of disease involvement, and 2) this approach would enable the use of robust machine learning algorithms to select robust biomarkers from large pool of genes and/or proteins.

EVs involved in intercellular signaling are a unique biological matrix comprising different biomolecules (e.g., DNA, RNA, proteins, and metabolites), and their compositions reflect the molecular and physiological status of the parental cell. Due to their high abundance in accessible body fluids, (Boukouris et al., “Exosomes in bodily fluids are a highly stable resource of disease biomarker,” Proteomics Clin. Appl. 2015: 9(3-4):358-67,) in the past decade, EVs have become a reliable source for biomarker discovery. (Id., Huda et al., “Potential Use of Exosomes as Diagnostic Biomarkers and in Targeted Drug Delivery: Progress in Clinical and Preclinical Applications,” ACS Biomater Sci. Eng. 2021: 7(6):2106-49.) However, most of the studies examining lung diseases have focused on the microRNA (miRNA) cargo within EVs for identifying biomarkers, (Njock et al., “Sputum exosomes: promising biomarkers for idiopathic pulmonary fibrosis,” Thorax 2019: 74(3):309-12,) while proteins have been less explored.

The present invention relates generally to a novel method for diagnosis and identification of interstitial pulmonary fibrosis (IPF). A protein signature from plasma EVs has been identified upon which diagnosis of IPF is based. The protein signature further distinguishes IPFs from other non-IPF interstitial lung diseases (ILDs) and healthy subjects. There has been no discovery on similar lines using exosomal proteins for IPF diagnosis.

### Certain Definitions

The term “idiopathic pulmonary fibrosis” or “IPF” as used herein refers to the most common type of pulmonary fibrosis which is a chronic and progressively fibrosing interstitial pneumonia of unknown etiology. IPF results in scarring of the lungs and causes irreversible and progressive lung damage.

The term “interstitial lung disease” or “ILD” as used herein refers to a group of diseases that cause progressive scarring of lung tissue. ILDs include, but are not limited to, IPF, chronic hypersensitivity pneumonitis (CHP), and nonspecific interstitial pneumonia (NSIP). The present disclosure includes references to “other ILDs,” i.e., ILDs excluding IPF.

The term “diagnosis” is used herein to refer to the identification or classification of a molecular or pathological state, disease or condition. For example, “diagnosis” may refer to identification of a particular type of ILD, such as IPF. The present disclosure include differentiating IPF patients from patients having other ILDs.

The term “aiding diagnosis” is used herein to refer to methods that assist in making a clinical determination regarding the presence, or nature, of a particular type of symptom or condition of IPF. For example, a method of aiding diagnosis of IPF can comprise measuring the expression of certain genes in a biological sample from an individual.

The term “high-resolution computed tomography” or “HRCT” is used herein to refer to a method of examination commonly used to diagnose lung diseases. HRCT is a crucial diagnostic component in clinical diagnosis of IPF. While IPF is indicated by a clear morphologic pattern of usual interstitial pneumonia (UIP) on HRCT, UIP is not synonymous with IPF as other ILDs, including CHP and NSIP, may also exhibit a similar pattern. (Wuyts et al.)

A “healthy subject” refers to a subject in good health who has not been diagnosed as having an ILD and not been diagnosed as having IPF.

As used herein, a “biomarker” is any gene or protein whose level of expression in a cell or tissue is altered in some way compared to that of a normal or healthy cell or tissue. In some aspects, the amount of biomarker may be changed. In other aspects, the biomarker may be differentially modified in some way. Biomarkers of the presently disclosed subject matter are selective for IPF. In some cases, proteins are listed as biomarkers but it is understood that the proteins themselves do not need to be detected but nucleic acids correlating to the proteins can be detected instead in the methods of the presently disclosed subject matter.

As used herein, the term “level of expression” of a biomarker refers to the amount of biomarker detected. Levels of biomarker can be detected at the transcriptional level, the translational level, and the post-translational level, for example.

The term “protein signature” is used interchangeably with “protein expression signature” and refers to one or a combination of proteins whose expression is indicative of a particular type of ILD or IPF characterized by certain molecular, pathological, histological, and/or clinical features. In certain aspects, the expression of one or more proteins comprising the protein signature is elevated compared to that in control subjects or a differential diagnosis population, e.g., elevated in IPF subjects relative to subjects having an ILD that is not IPF.

“SFTPB” refers to surfactant protein B, a protein which is encoded by the SFTPB gene in humans and which can serve as an exosomal protein biomarker.

“ALDOA” refers to Aldolase A or fructose-bisphosphate aldolase, a protein which is encoded by the ALDOA gene in humans and which can serve as an exosomal protein biomarker.

“HMGB1” refers to High mobility group box 1 protein, a protein which is encoded by the HMGB1 gene in humans and which can serve as an exosomal protein biomarker.

“CALML5” refers to calmodulin like 5, a protein which is encoded by the CALML5 gene in humans and which can serve as an exosomal protein biomarker.

“TLN1” refers to Talin-1, a protein which is encoded by the TLN1 gene in humans and which can serve as an exosomal protein biomarker.

A “biomarker panel,” as used herein, refers to a group of biomarkers that reflect different pathophysiological processes of a disease. In certain aspects, the biomarker panel refers to a group of biomarkers that are observed or analyzed in combination with each other. The biomarker panel may include the biomarkers in a kit. The kit may contain a plurality of the biomarkers in separate containers. The kit may contain instructions for performing a diagnosis method.

### Methods for Predicting or Diagnosing Idiopathic Pulmonary Fibrosis (IPF)

In one aspect, the presently disclosed subject matter provides a method for diagnosis and identification of interstitial pulmonary fibrosis (IPF) in a subject having IPF, at risk of having IPF, or suspected of having IPF, the method comprising: (a) performing proteomic analysis of plasma EVs in a human subject sample, (b) identifying a protein signature, and (c) validating the protein signature.

Various methods can be used to perform proteomic analysis of plasma EVs. Examples of such analytical methods include, but are not limited to, cryo-electron microscopy, nanoparticle tracking analysis, western blotting, liquid chromatography, mass spectrometry, transcriptome analysis, and statistical analysis.

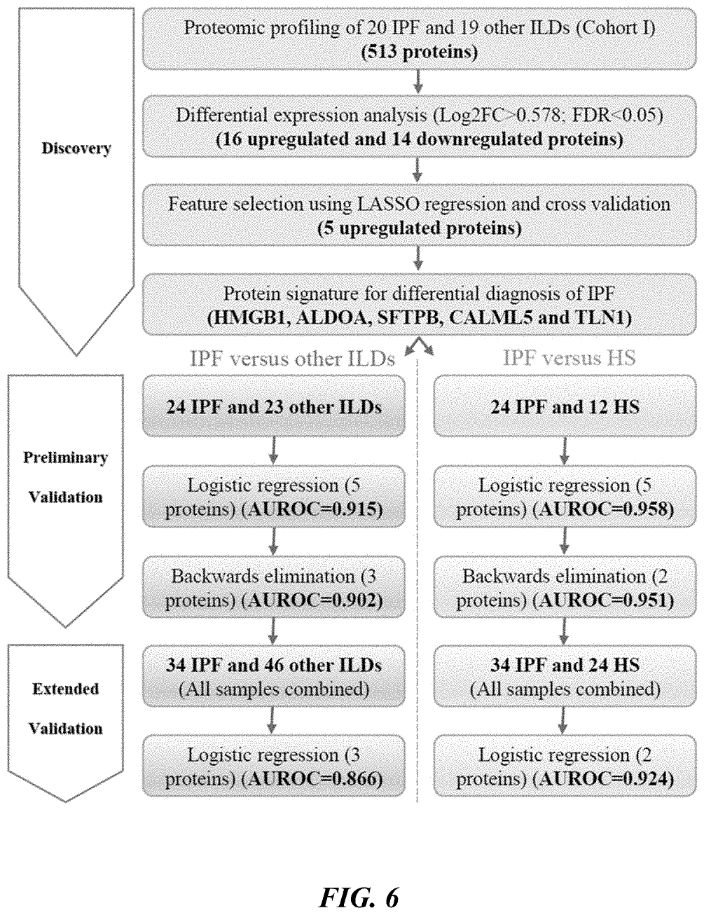

As illustrated in FIG. 6, mathematical modeling and computing can be used to identify a protein signature and validate the protein signature. Mass spectrometry based proteomic analysis was used for quantifying plasma EV proteins. Least Absolute Shrinkage and Selection Operator coupled with five-fold cross-validation was used for identifying a protein signature that discriminate IPF from other-ILDs. As part of preliminary validation, the signature was validated in an independent set of samples. To minimize the number of proteins in the signature, stepwise backwards elimination was used. The minimal signature was further tested on additional samples in extended validation where in additional samples as well as samples used for preliminary validation were combined to increase statistical power. The protein signature identified in discovery phase was also tested to discriminate IPF from HS in preliminary and extended validations identical the comparison of IPF and other-ILDs.

The protein signature can comprise multiple protein biomarkers. The protein biomarkers can comprise, for example, SFTPB, ALDOA, HMGB1, CALML5, and TLN1.

In some aspects, a definitive diagnosis cannot be made based on high-resolution computed tomography (HRCT). Although HRCT imaging can be used in some cases to diagnosis IPF by identifying a clear pattern of usual interstitial pneumonia (UIP), UIP is not synonymous with IPF as other interstitial lung diseases (ILDs), such as chronic hypersensitivity pneumonitis (CHP) and nonspecific interstitial pneumonia (NSIP), may also exhibit a similar pattern. Because several other ILDs often exhibit radiologic patterns similar to IPF on HRCT, diagnosis of the disease becomes difficult.

In some aspects, the method for diagnosis and identification of IPF can be implemented as an addition to HRCT and screening for IPF.

In another aspect, the presently disclosed subject matter provides a method for diagnosing and differentiating IPF from other ILDs using exosomal protein biomarkers. In a preferred aspect, the exosomal protein biomarkers comprise SFTPB, ALDOA, HMGB1, CALML5, and TLN1. In some aspects, a definitive diagnosis cannot be made based on HRCT. The method can be implemented as an addition to HRCT and screening for idiopathic pulmonary fibrosis.

In another aspect, the presently disclosed subject matter provides a method for screening for IPF in a noninvasive or minimally invasive manner using exosomal protein biomarkers. In a preferred aspect, the exosomal protein biomarkers comprise SFTPB, ALDOA, HMGB1, CALML5, and TLN1. In some aspects, a definitive diagnosis cannot be made based on HRCT. The method can be implemented as an addition to HRCT and screening for idiopathic pulmonary fibrosis.

In another aspect, the presently disclosed subject matter provides a method for diagnosing IPF comprising extracting a protein signature from plasma EVs.

In another aspect, the presently disclosed subject matter provides a diagnostic test for IPF comprising a protein biomarker panel. The protein biomarker panel can comprise exosomal protein biomarkers. In a preferred aspect, the exosomal protein biomarkers comprise SFTPB, ALDOA, HMGB1, CALML5, and TLN1. In some aspects, a definitive diagnosis cannot be made based on HRCT. The diagnostic test can be implemented as an addition to HRCT and screening for idiopathic pulmonary fibrosis.

In another aspect, the presently disclosed subject matter provides a biomarker panel for diagnosis of IPF in a noninvasive or minimally invasive manner. The biomarker panel can discriminate IPF from healthy subjects and other ILDs. The panel can comprise exosomal protein biomarkers. In a preferred aspect, the exosomal protein biomarkers comprise SFTPB, ALDOA, HMGB1, CALML5, and TLN1. The biomarker panel can be implemented as an addition to HRCT and screening for idiopathic pulmonary fibrosis.

While proteomic markers from serum or plasma have been reported in the past, their performance in differential diagnosis of IPF has been insufficient and unsatisfactory. In contrast, the present invention provides protein signatures that differentiate IPF from other non-IPF disease conditions with excellent specificity and sensitivity. In addition, the protein signature could also distinguish IPF from healthy control. Therefore, the invention is suitable for differential diagnosis as well as early diagnosis.

Diagnosis of IPF has been a challenging field and often requires a multidisciplinary approach involving pulmonologists, radiologists and pathologists with extensive expertise in the evaluation of patients with interstitial lung diseases (ILDs). Whilst the key features of IPF on HRCT or a multidisciplinary team consultation are important for accurate diagnosis of IPF, it needs to be acknowledged that access to these key elements of care in resource-constrained settings is a common challenge. Using the protein panel of the present disclosure, clinicians can differentiate IPF from other ILDs in resource limited settings and in situations when a definitive diagnosis cannot be made based on HRCT.

The majority of prior studies evaluated selected genes/proteins with a putative role in the pathology of the ILDs for their efficacy as diagnostic markers, but their diagnostic efficacy has been unsatisfactory. In contrast, the specificity and sensitivity of the 5-protein biomarker panel for differential diagnosis of IPF of the present invention are 91.3% and 91.7%, respectively. Similarly, for discriminating IPF from healthy subjects, specificity and sensitivity of the panel of the present disclosure are 96% and 92%, respectively. These efficacy values are the highest compared to previously known results.

Idiopathic pulmonary fibrosis (IPF) is a fibrosing interstitial pneumonia of unknown etiology often leading to respiratory failure. Over half of IPF patients present with discordant features of usual interstitial pneumonia on high-resolution computed tomography at diagnosis which warrants surgical lung biopsy to exclude the possibility of other interstitial lung diseases (ILDs). Therefore, there is a need for non-invasive biomarkers for expediting the differential diagnosis of IPF. Using mass spectrometry, we performed proteomic analysis of plasma EVs in a cohort of subjects with IPF, chronic hypersensitivity pneumonitis, nonspecific interstitial pneumonitis, and healthy subjects (HS). A five-protein signature was identified by lasso regression and was validated in an independent cohort using ELISA. We evaluated the concordance between plasma EV proteome and the lung transcriptome data. Lastly, we compared the molecular pathways overrepresented in IPF by differentially expressed proteins and transcripts from EVs and lung tissues, respectively. The five-protein signature derived from mass spectrometry data showed area under the receiver operating characteristic curve of 0.915 (95% CI: 0.819-1.011) and 0.958 (95% CI: 0.882-1.034) for differentiating IPF from other ILDs and from HS, respectively. We also found that the EV protein expression profiles mirrored their corresponding mRNA expressions in IPF lungs. Further, we observed an overlap in the EV proteome- and lung mRNA-associated molecular pathways. The present disclosure includes a novel and inventive plasma EV-based protein signature for differential diagnosis of IPF and disclosures of its validation in an independent cohort.

A clear morphologic pattern of usual interstitial pneumonia (UIP) on high-resolution computed tomography (HRCT) with the absence of features that support alternate diagnosis confirms IPF, while discordant UIP features (detected in over 50% patients) warrants histopathological analysis of surgical lung biopsies for definitive diagnosis. However, surgical lung biopsy is not recommended for patients who are prone to high risk complications owing to their advanced age and poor fitness, and therefore the contemplation thwarts a confident diagnosis. Additionally, UIP is not synonymous with IPF as other interstitial lung diseases (ILDs) including chronic hypersensitivity pneumonitis (CHP), and nonspecific interstitial pneumonia (NSIP), etc., may also exhibit a similar pattern. Acquisition of adequate and faithfully representative biopsy sample is essential for accurate diagnosis of IPF and exclusion of other ILDs. Confirmation of IPF often requires a multidisciplinary approach involving pulmonologists, radiologists and pathologists with extensive expertise in the evaluation of patients with ILDs. Whilst the key features of IPF on HRCT or a multidisciplinary team consultation are important for accurate diagnosis of IPF, it needs to be acknowledged that access to these key elements of care in resource-constrained settings is a common challenge. Therefore, in some cases of IPF, a significant diagnostic delay is reported that is mainly attributable to the patients, general practitioners, and community hospitals. Therefore, there is an unmet need for a biomarker or a biomarker panel that can be used in a laboratory to permit and expedite the differential diagnosis of IPF.

Several previous studies have reported biomarkers for differential diagnosis of IPF. The majority of the studies evaluated selected genes/proteins with a putative role in the pathology of the ILDs for their efficacy as diagnostic markers, but their diagnostic efficacy has been unsatisfactory. As the molecular changes in IPF pathogenesis are complex, biomarker discovery using high throughput ‘omics’ approaches may yield robust biomarkers.

EVs involved in intercellular signaling, contain different biomolecules (DNA, RNA, proteins, and metabolites) and their compositions reflect the molecular and physiological status of the parental cell. Due to their abundance in accessible body fluids, in the past decade, EVs have gained prominence as a potential matrix for discovery of biomarkers of disease. However, most of the studies examining lung diseases have focused on the miRNA cargo within EVs for identifying biomarkers, while proteins have been less explored.

### EXAMPLES

The following Examples have been included to provide guidance to one of ordinary skill in the art for practicing representative aspects of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Collection of Plasma Samples

A total of 163 human plasma samples including 54 IPF, 38 CHP, 27 NSIP and 44 healthy subject (HS) samples were collected from four different ILD centers and biobanks. Plasma samples were collected from subjects at the time of diagnosis or during one of the follow-up clinical visits. Specifically, peripheral blood was collected from participants in EDTA tubes or Citrated CPT tubes after informed consent. Plasma was separated from blood no later than 1 hour from the time of collection. Blood samples were centrifuged at 1100×g for 10 minutes at room temperature and stored in −80° C.

There was no significant difference between non-IPF and IPF samples with respect to age, gender, and lung function parameters. Demographic and clinical features of the subjects participating in the study are set forth in Table 1.

TABLE 1

| | | Other ILDs | | | | |
|---|---|---|---|---|---|---|
| | IPF | CHP | NSIP | HS | P value^{a} | |
| Total (N) | 54 | 38 | 27 | 20 | IPFs vs | IPF vs HS |
| | | | | | other | |
| | | | | | ILDs | |

| | |
|---|---|
| — | — |

Demographic information

| Age (Years) | | 66.9 (43- | 62.3 (32- | 64.3 (27- | 64.6 (57- | 0.0853 | 0.1014 |
|---|---|---|---|---|---|---|---|
| | | 81) | 81) | 82) | 80) | | |
| Gender | Male | 36 | 22 | 14 | 29 | 0.2595 | 1 |
| (N) | Female | 18 | 16 | 13 | 15 | | |
| Smoking | Ever | 27 | 12 | 6 | 21 | 0.1851 | 0.5023 |
| (N) | Never | 16 | 11 | 9 | 18 | | |
| | NA | 11 | 15 | 12 | 5 | | |

Lung function parameters

| | | | | | | |
|---|---|---|---|---|---|---|
| FEV1% | 79 (36.5- | 77.5 (37- | 72.8 (32- | — | 0.3802 | — |
| | 129.0) | 127.5) | 116.6) | | | |
| FVC % | 67.3 (31- | 71.4 (37- | 67.8 (30.9- | — | 0.5138 | — |
| | 99.6) | 123.2) | 100.3) | | | |
| FEV1/FVC | 83.9 (29- | 90.5 (55- | 88.1 (69.4- | — | 0.3006 | — |
| | 120) | 122) | 120.7) | | | |

^{a}Student t test was used for age, FEV1, FVC, and FEV1/FVC; Chi-square test was used for gender and smoking.

### Example 2

### Preparation of EV Samples

Extracellular vesicles were isolated from blood plasma samples in cohort-I consisting of 20 IPF, 19 non-IPF (11 CHP and 8 NSIP), and 20 HS samples from the University of Pittsburgh, Pittsburgh, USA and the Brigham and Women's Hospital, Boston, USA, which were prepared in Example 1 above. Size exclusion chromatography (SEC) was performed for isolating EVs using qEV original 35 nm pore size columns and an automated fraction collector V1 setup (Izon Science US Ltd, MA, USA), from 150 μL of plasma, as per the manufacturer's instructions. Plasma was clarified by centrifugation at 2500×g for 15 minutes and subsequently at 10000×g for 10 min at 4° C. SEC columns were equilibrated with phosphate buffered saline, and clarified plasma was loaded onto the column. After discarding 3 mL of void volume, 2 ml of EVs were collected and concentrated using 300 KDa centrifugal filters (Pall Corp., NY, USA) at 3500×g at 4° C.

### Example 3

### Characterization and Proteomic Landscape of Plasma EVs

Various analysis methods were used to characterize and identify the proteomic landscape of the plasma EVs isolated in Example 2.

A. Cryo-Electron Microscopy

Four microliters of the EV solution (1:20 dilution) was added to Lacey carbon grids (200-mesh; Electron Microscopy Sciences) that were negatively glow-discharged for 30 s at 30 mA. Excess sample was removed by blotting once about every 2 seconds with Whatman filter paper, and then the grid was plunge-frozen in liquid ethane cooled by liquid nitrogen using a homemade plunge-freezer. The vitrified vesicle samples were imaged using a Titan Krios 300 kV transmission electron microscope (FEI, Hillsboro, Oreg., USA) equipped with a post-column Gatan imaging filter (Gatan Inc, Pleasanton, Calif., USA) and a Volta Phase Plate (FEI). The SerialEM software was used to collect 2D images and tilt series under low-dose conditions. Medium magnification maps (MMMs) and high magnification maps (HMMs) were recorded at 2250× magnification and 42000× magnification, respectively, without using dose fractionation. Tilt series were recorded at 42000× magnification on a K3 Summit direct electron detector (Gatan Inc, Pleasanton Calif., USA) with an effective pixel size of 0.99 Å in dose fractionation mode. For each image, 20 frames were recorded over is exposure time at a dose rate of 26.03 electrons/pixel/s. The defocus was set to −0.5 μm (with phase plate) and the energy filter was in zero-loss mode with a slit width of 20 eV. The movie frames were aligned using MotionCorr2. (Furusawa et al., “Chronic Hypersensitivity Pneumonitis, an Interstitial Lung Disease with Distinct Molecular Signatures,” Am. J. Respir. Crit. Care Med. 2020: 202(10):1430-44.)

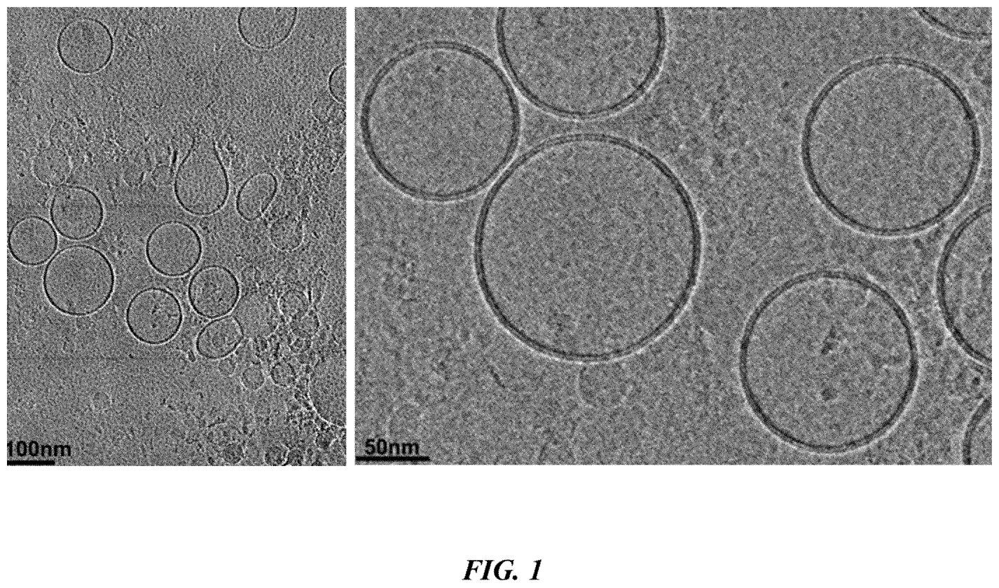

Cryo-electron microscopy imaging of plasma EV preparations revealed the presence of intact spherical vesicles consisting of a lipid bi-layer and were on average 100 nm in diameter (FIG. 1).

B. Nanoparticle Tracking Analysis

EV concentration and size were determined via nanoparticle tracking analysis using a Zetaview Quatt instrument (Particle Metrix, Germany) in scatter mode with a 520 nm laser and sCMOS camera. The instrument was calibrated using 100 nm fluorescent polystyrene beads. EVs were diluted using filtered PBS. Data acquisition was performed at the following parameters: Sensitivity=80; Shutter=100; Track length=15; Minimum brightness=20; Cycles/position=2/11.

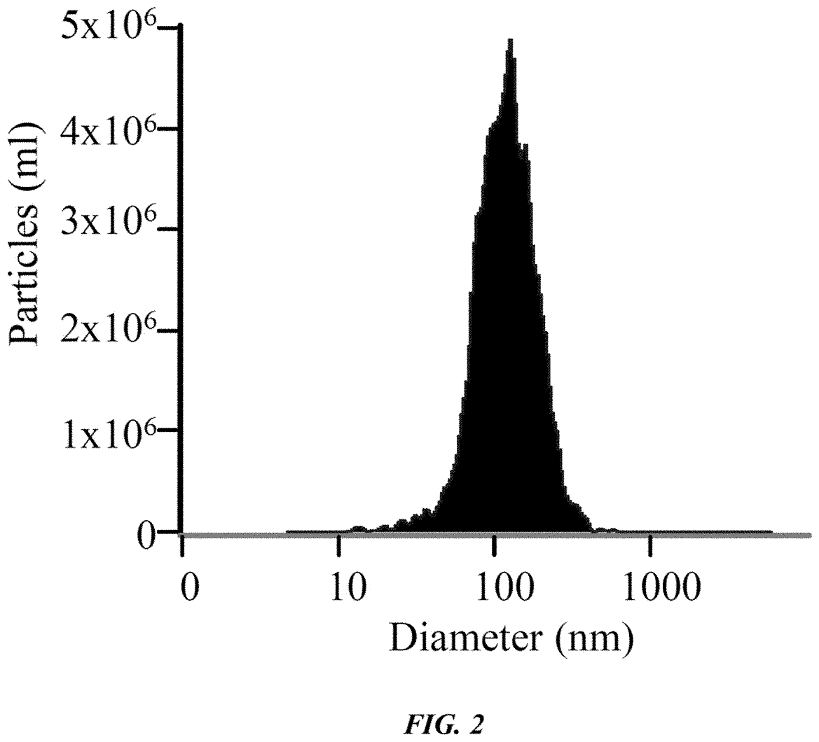

The majority of EVs were in the range of 50-200 nm in diameter (FIG. 2).

C. Lysis of EVs, Protein Estimation, and Western Blot

The EV fractions from SEC were concentrated down to 50 μL in vacuum at 30° C., lysed in RIPA buffer (Sigma Aldrich, USA) at 95° C. for 10 min, and sonicated in an ultrasonic sonicator bath (Branson Ultrasonics Corp, CT, USA) for 2 min. The supernatants containing the EV proteins were collected after centrifugation at 30000 g for 15 min at 4° C. The protein concentration was estimated using a Pierce BCA protein assay kit (Thermo Fisher Scientific, MA, USA), using bovine serum albumin as standard. A western blot was performed following SDS PAGE using standard protocols involving TSG101 (Cat#14497) and CD81 (Cat#66866) antibodies from Protein Tech, Rosemont, Ill., USA; CD9 (Cat# ab195422) antibodies from Abcam; HSP70 (Cat# SC-24); albumin (Cat# SC-271605); and calnexin (Cat# SC-23954) antibodies from Santa Cruz Biotechnology, CA, USA. Proteins were detected by chemiluminiscence using a Clarity ECL substrate (Bio-Rad Laboratories, CA, USA) on a Chemidoc MP Gel Imaging System (Bio-Rad Laboratories). All EV markers were exposed for a short duration (2-30 seconds), while the anti-albumin blot was exposed for longer (2 minutes) in order to detect clearer, brighter bands.

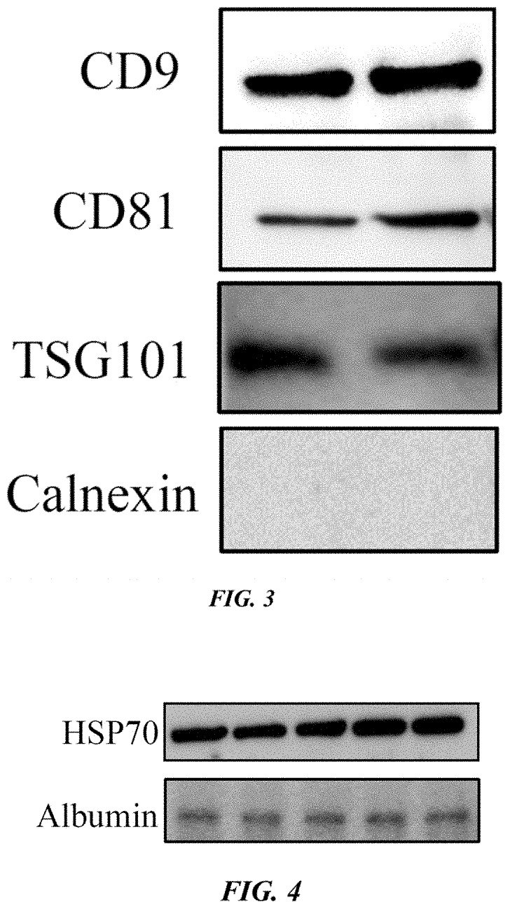

The tetraspanin protein CD9 and other exosomal proteins such as Cd81 and TFG101 were detected. Meanwhile, calnexin, an endoplasmic reticulum membrane protein, was absent in EV preparation, suggesting that the isolated EVs were not contaminated with other cellular organelles (FIG. 3). The results demonstrated that the plasma EV preparations were enriched with exosomes, and EV preparations exhibited low and similar background levels of plasma protein contamination (e.g., albumin levels were similar across samples (FIG. 4)).

D. Liquid Chromatography—Mass Spectrometry Analysis

Dried peptide samples were dissolved in 4.8 μL of 0.25% formic acid with 3% (vol/vol) acetonitrile, and 4 μL of each sample was injected into an Easy-nLC 1000 (Thermo Fisher Scientific). Peptides were separated on a 45-cm in-house packed column (360 μm OD×75 μm ID) containing C18 resin (2.2 μm, 100 Å; Michrom Bioresources, CA, USA). The mobile phase buffer consisted of 0.1% formic acid in ultrapure water (buffer A) with an eluting buffer of 0.1% formic acid in 80% (vol/vol) acetonitrile (buffer B) run with a linear 60-min gradient of 6-30% buffer B at flow rate of 250 nL/min. The Easy-nLC 1000 was coupled online with a hybrid high-resolution LTQ-Orbitrap Velos Pro mass spectrometer (Thermo Fisher Scientific). The mass spectrometer was operated in the data-dependent mode, in which a full-scan mass spectrometry (MS) (from m/z 300 to 1,500 with the resolution of 30000 at m/z 400) was performed, followed by MS/MS of the 10 most intense ions (normalized collision energy—30%; automatic gain control (AGC)—3E4; maximum injection time—100 ms; 90 s exclusion). The raw files were searched directly against the human Uniprot database version (downloaded in August 2017) with no redundant entries, using the Byonic search engine (Protein Metrics, CA, USA) loaded into the Proteome Discoverer 2.2 software (Thermo Fisher Scientific). The initial precursor mass tolerance was set at 10 ppm, the final tolerance was set at 6 ppm, and ion trap mass spectrometry (ITMS) MS/MS tolerance was set at 0.6 Da. The search criteria included a static carbamidomethylation of cysteines (+57.0214 Da), and variable modifications of oxidation (+15.9949 Da) on methionine residues and acetylation (+42.011 Da) at N terminus of proteins. The search was performed with full trypsin/P digestion and allowed a maximum of two missed cleavages on the peptides analyzed from the sequence database. The false-discovery rates of proteins and peptides were set at 0.01. All protein and peptide identifications were grouped, and any redundant entries were removed. Only unique peptides and unique master proteins were reported.

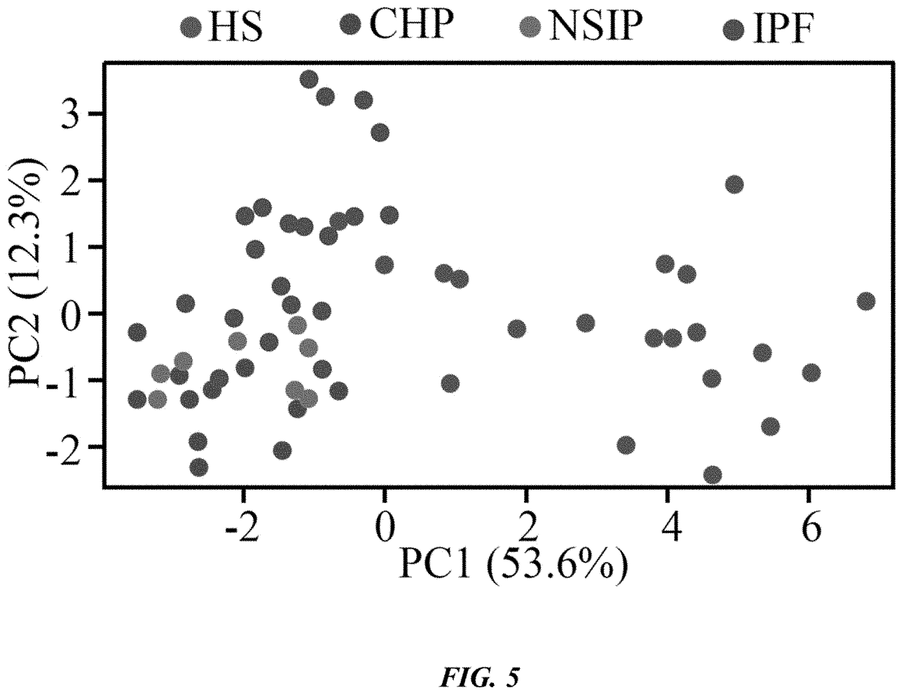

Mass spectrometry-based proteomic profiling of EVs from cohort-I quantified the expression of 520 proteins. Principle component analysis revealed that the protein profiles of EVs from healthy subjects, CHP, NSIP, and IPF clustered according to the lung conditions (FIG. 5). HS samples appeared distinct from all of the ILDs. While the three ILDs clustered separately, they exhibited major overlap, underscoring the similarities in the proteome profiles among different ILDs.

E. Transcriptome Analysis

For comparing transcriptomic changes in lung tissue of CHP and IPF with healthy subjects raw read counts of a large RNA sequencing data from a previous study (GSE150910) were used. (Furusawa et al.) 103 IPF samples and 103 HS samples were analyzed. Raw read counts of transcripts were collapsed to gene IDs using tximport package. Data transformations, normalization and differential expression analysis were carried out using DESeq2. (Love et al., “Moderated estimation of fold change and dispersion for RNA-seq data with DESeq2,” Genome Biol. 2014: 15(12):550.) Genes showing a minimum log 2 fold change of 1 at FDR<0.01 were considered differentially expressed. Pathway analysis was performed on differentially expressed genes using g:profiler package. (Raudvere et al., “g:Profiler: a web server for functional enrichment analysis and conversions of gene lists (2019 update),” Nucleic Acids Res. 2019: 47(W1):W191-W198.)

F. Statistical Analysis

All statistical analyses were performed in the R environment (R4.0.3) if not stated otherwise. A student t-test was used to compare the means of two groups. A chi-square test was used to compare categorical variables, and a student t-test was used to compare continuous variables. P value<0.05 was considered statistically significant.

### Example 3

### Biomarker Discovery and Validation

Biomarker discovery was performed on cohort-I samples prepared in Example 1 and analyzed in Example 2. A schematic of steps for biomarker discovery and the validation pipeline is shown in FIG. 6. To identify biomarkers that distinguish IPF from other ILDs, differential expression analysis was performed on the proteome profiles of EVs isolated from the plasma samples corresponding to 20 IPF patients and a total of 19 patients with other ILDs. The other ILD patients' samples included 11 CHP and 8 NSIP samples exhibiting fibrosis that served as ILD controls to identify proteins specific to IPF. A total of 30 differentially expressed genes were identified at FDR and llog2 fold changel cut offs of 0.05 and 0.585, respectively, as set forth in Table 2 below. Protein abundance values obtained from mass spectrometry were used to determine relative protein amounts between samples. (Zhao et al., “Comparative evaluation of label-free quantification strategies,” J. Proteomics 2020: 215:103669; Ramirez-Martinez et al., “The nuclear envelope protein Net39 is essential for muscle nuclear integrity and chromatin organization,” Nat. Commun. 2021: 12(1):690; Behrmann et al., “PTH/PTHrP Receptor Signaling Restricts Arterial Fibrosis in Diabetic LDLR(-/-) Mice by Inhibiting Myocardin-Related Transcription Factor Relays,” Circ. Res. 2020: 126(10):1363-78.) The protein abundance values were log₂(n+1) transformed prior to differential expression analysis. Based on the premise that upregulated proteins as biomarkers are more suitable for diagnostic use in clinical settings and with the aim to develop an easy-to-adopt assay, proteins detected in less than 10% of the samples were removed from further analysis, and upregulated proteins were prioritized for further analysis. A t-test was used for identifying differentially expressed genes between different classes of samples, using an FDR cut off value of 0.05 to correct for multiple hypothesis testing. Proteins were considered differentially expressed if both (A) the t-test indicates significant differential expression and (B) llog₂ fold changel≥0.585 (i.e., equivalent to 50% increase or decrease in expression). Using a Glmnet package, (Friedman et al., “Regularization Paths for Generalized Linear Models via Coordinate Descent,” J. Stat. Softw. 2010: 33(1):1-22,) Least Absolute Shrinkage and Selection Operator (LASSO) regression was applied to mass-spectrometry protein abundances of upregulated proteins, coupled with five-fold cross-validation to minimize prediction error, in order to identify a robust biomarker panel. The five-fold cross validation was performed using a cv.glmnet function to estimate cross validation error at varying values of tuning parameter (κ). Features included in the binomial model at λₘᵢₙ were selected as the protein signature.

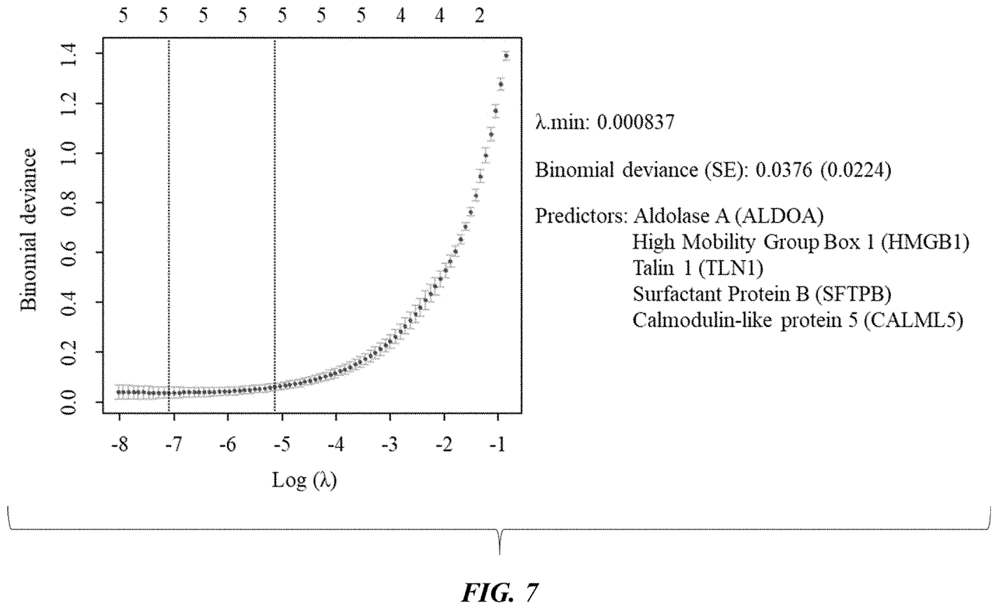

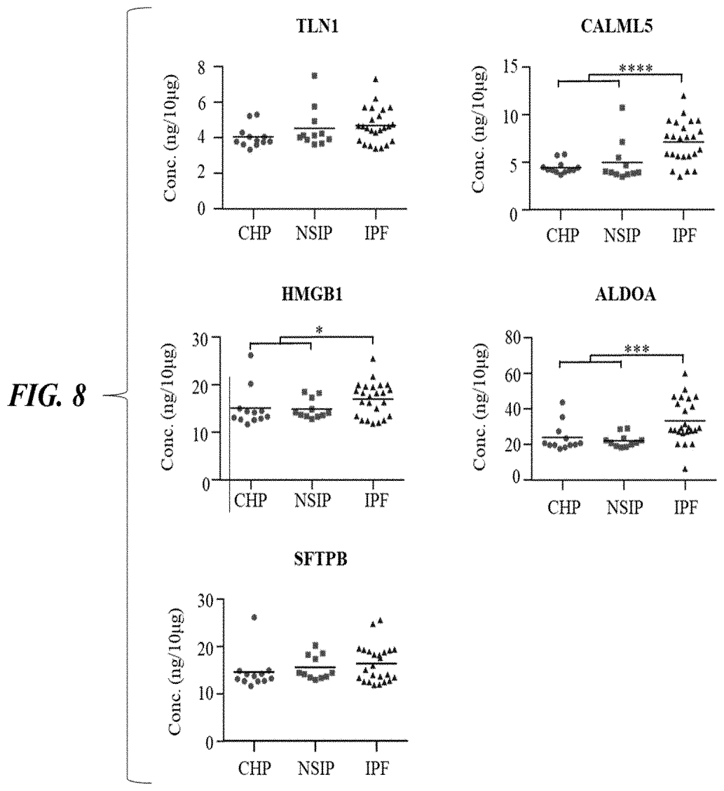

At a minimum value of λ (0.000837), a five-protein signature comprising High mobility group box protein 1 (HMGB1), surfactant protein B (SFTPB), Aldolase A (ALDOA), calmodulin like 5 (CALML5), and Talin-1 (TLN1) discriminated IPF from other ILDs with minimum cross validation error (FIG. 7).

TABLE 2

| Protein symbol | FDR(BH) | Log2FC |
|---|---|---|
| CEP290 | 0.031835012 | −3.67489 |
| PTPRG | 0.013379933 | −2.42574 |
| DCDC1/DCDC5 | 0.006411218 | −2.28237 |
| UTP14C | 0.047131114 | −1.78836 |
| CRTAC1 | 0.006411218 | −1.12103 |
| EMD | 0.006411218 | −1.07538 |
| ATRN | 0.034193161 | −0.92917 |
| SDF4 | 0.006411218 | −0.77978 |
| MT-CO2 | 0.006411218 | −0.59182 |
| FLG2 | 0.01079784 | 0.650379 |
| TXN | 0.044878524 | 0.748756 |
| MASP1 | 0.029308424 | 0.780045 |
| VWF | 0.039708187 | 0.810987 |
| DSG1 | 0.01079784 | 0.819757 |
| CSTA | 0.047131114 | 0.835759 |
| AEDOA | 0.036208758 | 1.122451 |
| SFTPD | 0.018996201 | 1.146431 |
| A2ML1 | 0.046627038 | 1.228911 |
| LCN1 | 0.013379933 | 1.440185 |
| ARG1 | 0.006411218 | 1.643993 |
| HMGB1 | 0.006411218 | 1.714167 |
| LAMA2 | 0.018996201 | 1.78414 |
| SDR9C7 | 0.017096581 | 1.985864 |
| ALOX12B | 0.047131114 | 2.036869 |
| CALML5 | 0.01079784 | 2.070101 |
| TLN1 | 0.048590282 | 2.342485 |
| SERF2 | 0.006411218 | 2.564957 |
| TPP1 | 0.018996201 | 2.575143 |
| SFTPB | 0.006411218 | 3.83857 |
| PRRC2C | 0.006411218 | 3.916179 |

The biomarker signature was then validated in preliminary and extended validation steps on cohort-II samples prepared in Example 1 comprising 34 IPF, 27 CHP, 19 NSIP and 24 HS samples from Hiroshima University, Hiroshima, Japan; National Heart, Lung, and Blood Institute (NHLBI), Bethesda, USA; and Brigham and Women's Hospital, Boston, USA.

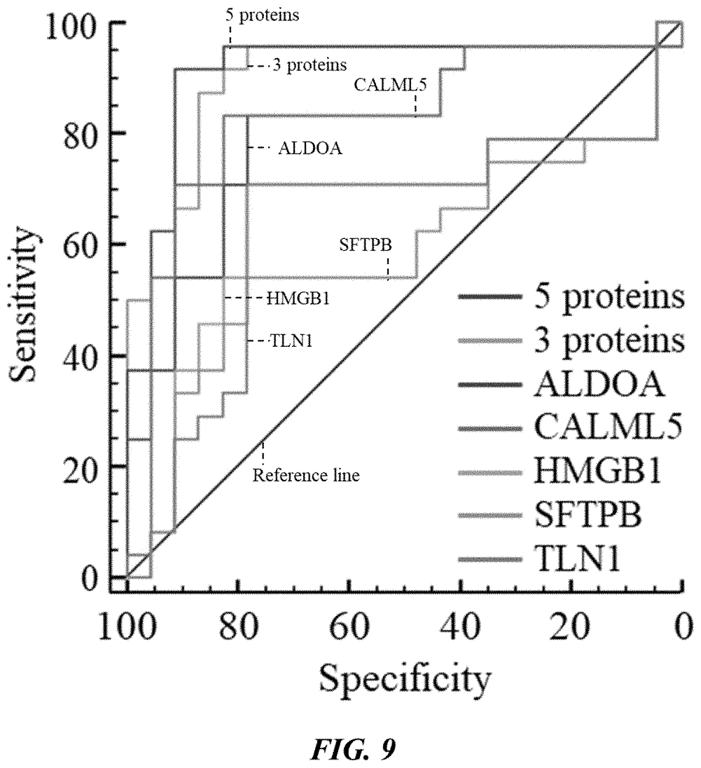

Preliminary biomarker validation was performed using an enzyme-linked immunosorbent assay (ELISA) in order to estimate the levels of the protein biomarkers identified above in the plasma EVs of 24 IPF, 23 other ILDs, and 12 HS samples from a validation cohort independent of the samples analyzed in Example 2. ELISA was performed on standard sandwich ELISA kits (Aviva Systems Biology, CA, USA) as per the manufacturer's instructions, and the microplates were read using a Synergy microplate reader (BioTek Instruments Ltd., VT, USA). The expression levels of HMGB1, ALDOA and CALML5 were found to be different between IPF and other ILDs in an independent cohort (FIG. 8). To evaluate the efficacy of the five EV proteins for differential diagnosis of IPF, an SPSS package was used for constructing binomial logistic regression models and ROC curves (FIG. 9). The logistic regression classifier was further evaluated by plotting calibration curves and calculating the concordance index. Individual risk scores were calculated for each subject in the logistic regression model by subtracting 0.5 from predicted probability value of the subject. The identified protein signature classified IPF and other ILDs with excellent efficacy in independent samples (AUROC=0.915, 95% CI: 0.819-1.011) (FIG. 9). A backward stepwise elimination approach was applied to check whether the number of proteins in the classifier could be minimized, and a sparse model was selected comprising CALML5, HMGB1 and TLN1 which exhibited an AUROC of 0.902 (95% CI:0.805-1) (FIG. 9).

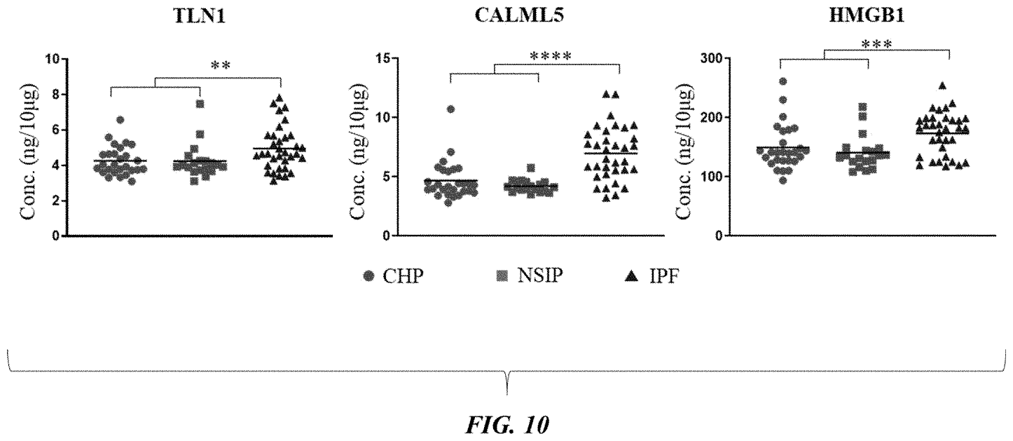

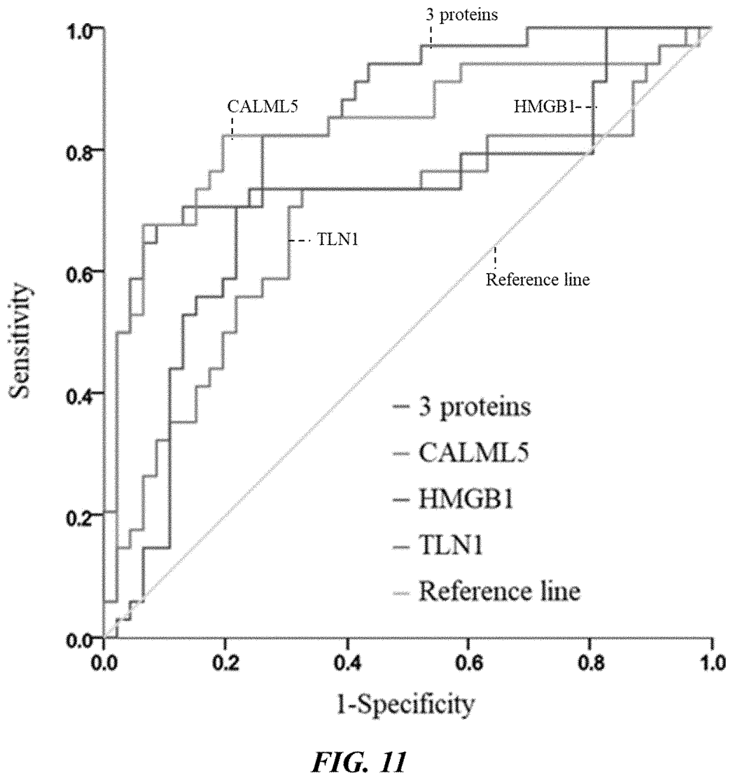

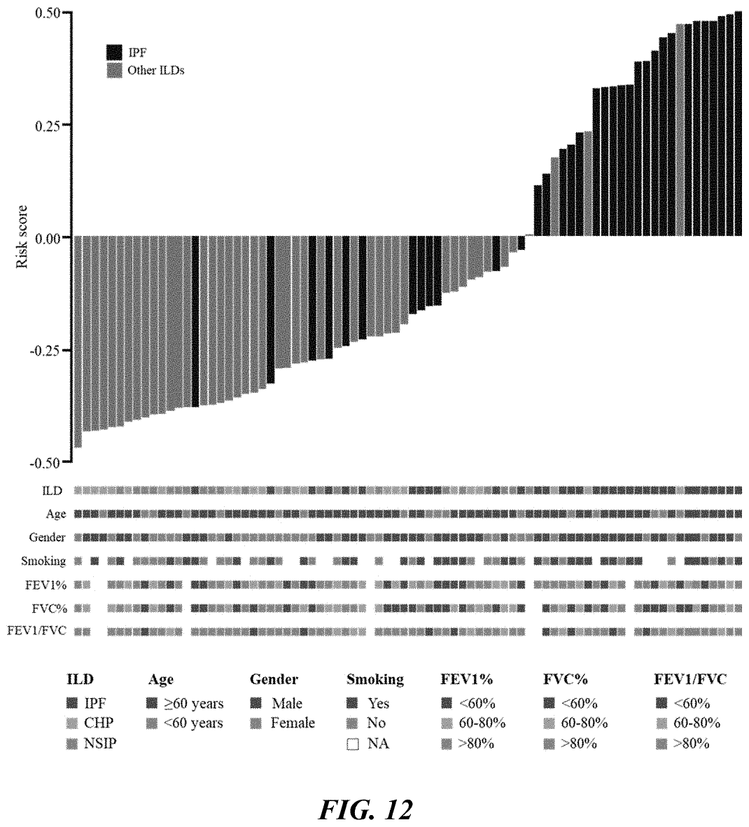

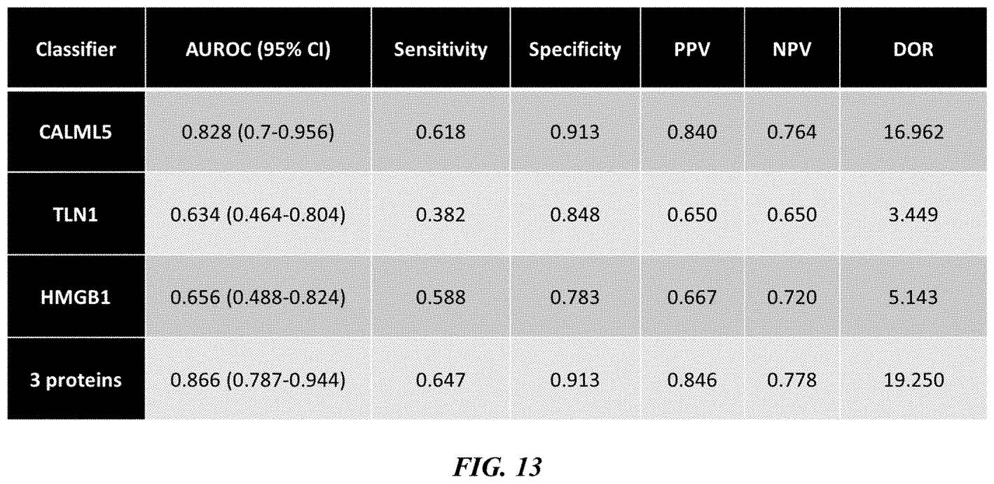

Next, extended validation of this three-protein signature was performed on additional samples comprising 10 IPF, 15 CHP and 8 NSIP samples (FIG. 10). When logistic regression was performed for all samples together (including those from the preliminary validation), the three-protein signature showed an AUROC of 0.866 (0.787-0.944) (FIG. 10, FIG. 11, FIG. 13). Taken together, these results suggest that the identified EV protein biomarker panel has superior efficacy in differential diagnosis of IPF.

### Example 4

### EV Protein Biomarkers for Discriminating IPF from Healthy Subjects

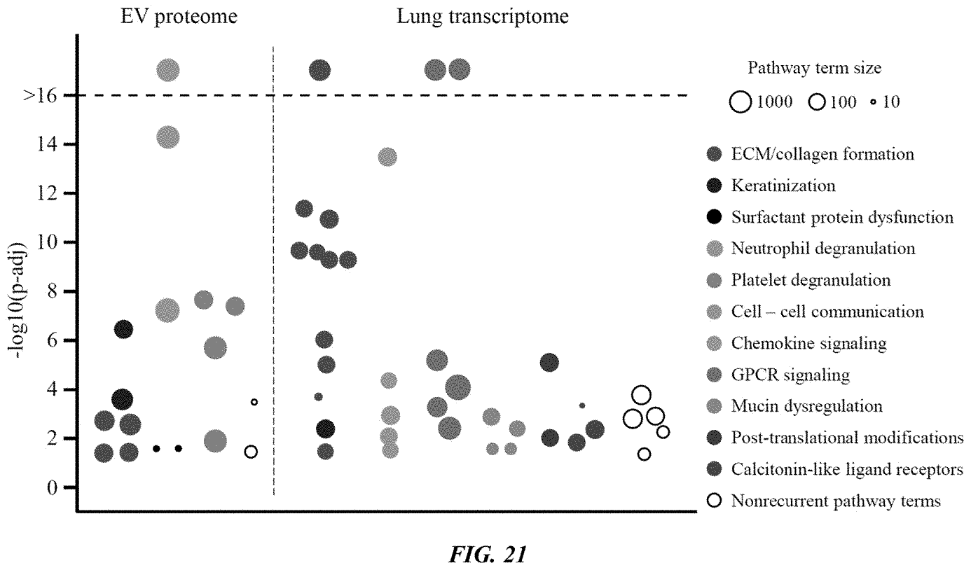

Differentially expressed proteins (from plasma EV proteome) and genes (from tissue transcriptome) were subjected to overrepresentation analysis separately. Extracellular matrix and collagen deposition were commonly identified by EV proteome and lung transcriptome. Notably, EV proteome identified key IPF molecular events such as neutrophil and platelet degranulation and surfactant protein dysfunction, while transcriptome did not (FIG. 21). However, lung transcriptome identified several additional pathways, including G-protein couple receptor signaling, chemokine signaling, and mucin dysregulation (FIG. 21).

Definitive diagnosis of IPF is often challenging because a few other fibrotic lung diseases also exhibit pathological features similar to IPF. The present disclosure identifies a novel protein signature, consisting of exosomal protein biomarkers, for differential diagnosis of IPF from other ILDs with greater specificity and sensitivity than has been demonstrated in the field thus far. Additionally, mass spectrometry- and ELISA-based systematic discovery and development of plasma EV protein biomarkers is described herein.

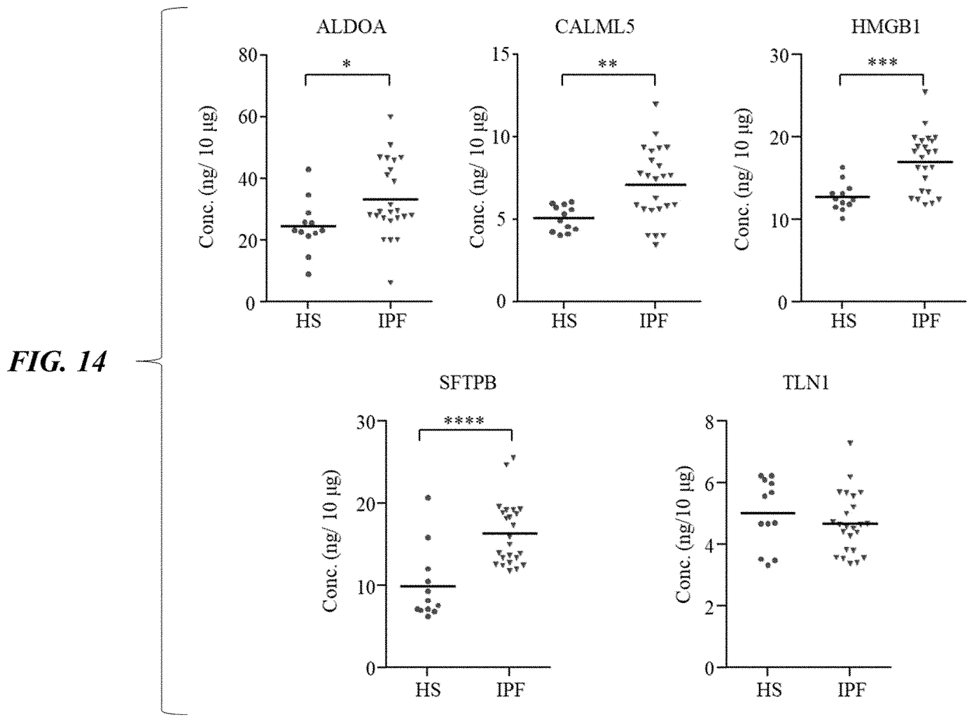

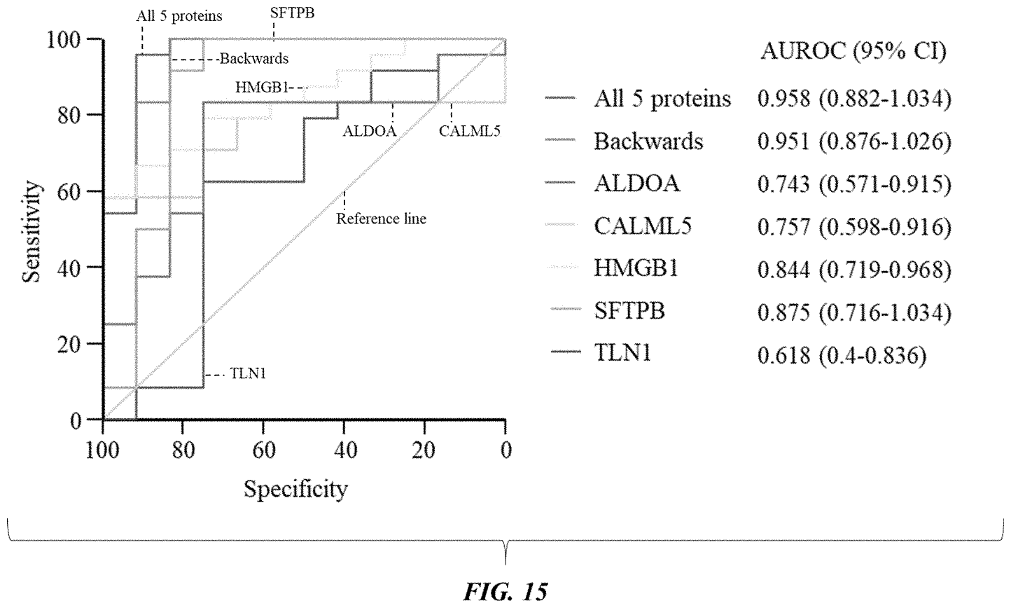

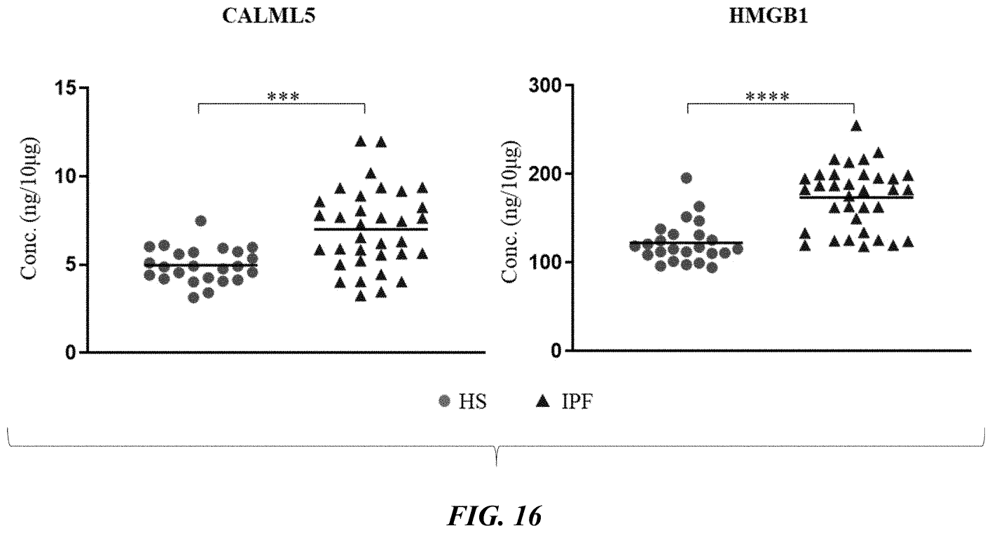

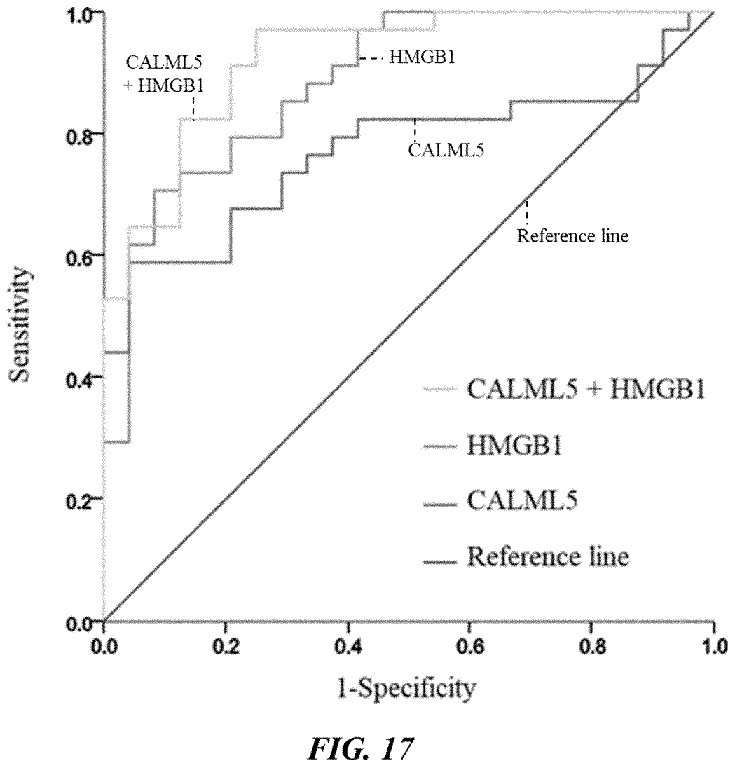

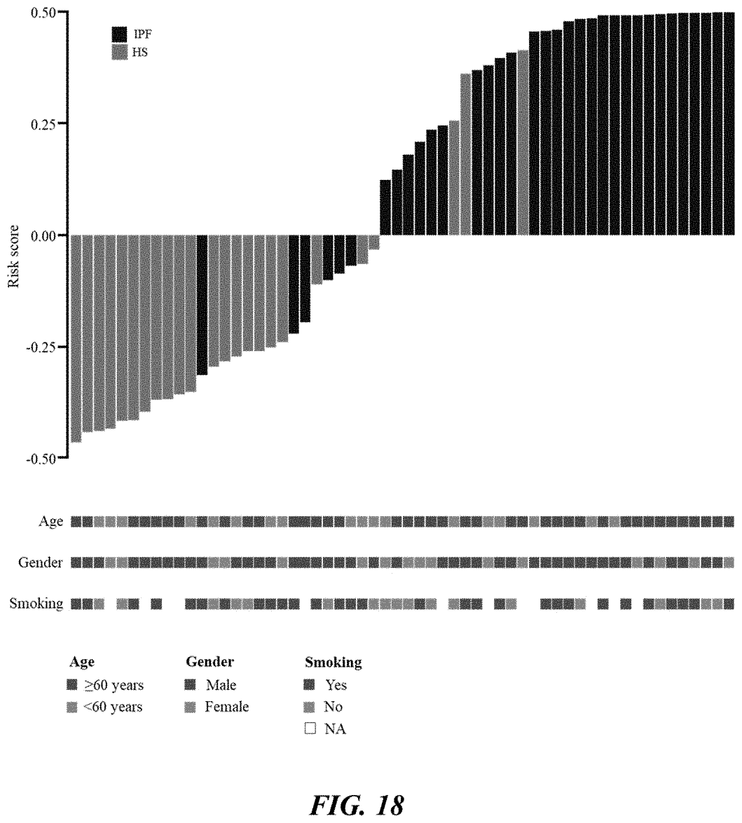

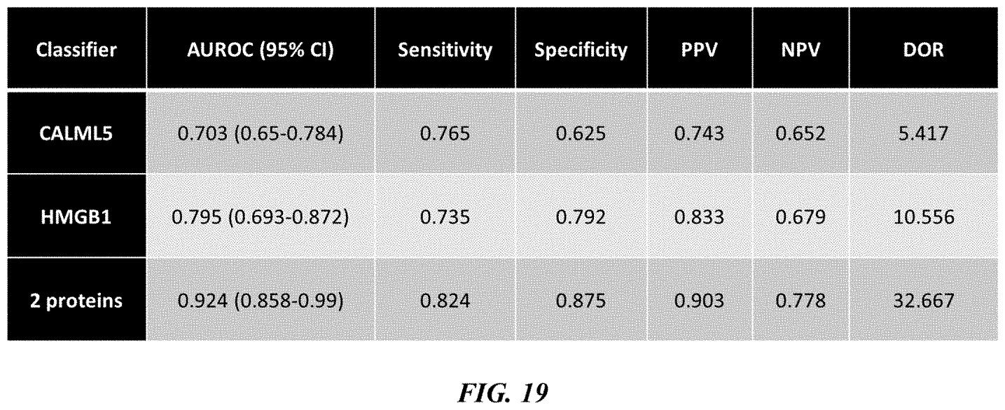

As shown in FIG. 14, three out of the five proteins identified in Example 3, namely CALML5, HMGB1, and SFTPB, exhibited significantly high expression in IPF samples compared to in HS samples. Validation steps were performed in order to investigate if the five-protein biomarker signature was suitable for differentiating IPF from HS. As part of preliminary validation, a logistic regression model and ROC curves were constructed, revealing that the five-protein signature could distinguish IPF from healthy controls with good accuracy (AUROC=0.958) (FIG. 15). A backward stepwise elimination approach yielded a model comprising CALML5 and HMGB1 which exhibited an AUROC of 0.951 (95% CI:0.876-1.026) (FIG. 15). In extended validation, the two proteins CALML5 and HMGB1 were quantified in an additional 10 IPF and 12 HS samples (FIG. 16). When logistic regression was performed on all samples together (including those in preliminary validation), the two proteins showed an AUROC of 0.924 (0.858-0.99) (FIG. 17, FIG. 18). Taken together, these results suggest that the EV biomarkers have excellent efficacy in discriminating IPF from HS.

### Example 5

### EV Proteome Identifying Deregulated Signaling Pathways in IPF

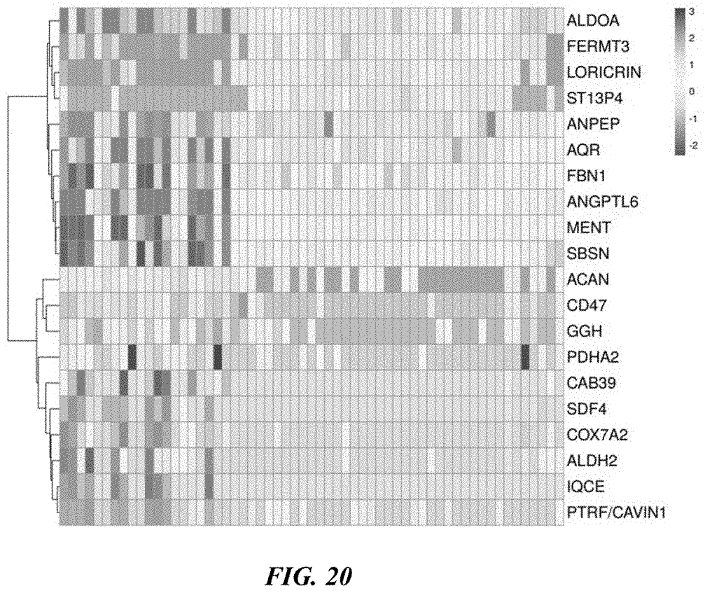

A comprehensive analysis of transcriptome profiles has been explored previously to identify potential pathways involved in the pathogenesis of IPF. (Furusawa et al., “Chronic Hypersensitivity Pneumonitis, an Interstitial Lung Disease with Distinct Molecular Signatures,” Am. J. Respir. Crit. Care Med. 2020: 202(10):1430-44; Yang et al., “Expression of cilium-associated genes defines novel molecular subtypes of idiopathic pulmonary fibrosis,” Thorax 2013: 68(12):1114-21.) The plasma EV proteome was analyzed to determine whether it could reveal dysregulated pathways in IPF, and the enriched pathways were compared with those identified from lung tissue transcriptome. In order to do so, first, differential expression analysis was performed for IPF and healthy subjects. The EV proteome of IPF patients revealed 154 upregulated and 32 downregulated proteins that were identified at an FDR<0.05 and llog2 fold changel>0.585, as set forth in Table 3 below. Four (HMGB1, SFTPB, ALDOA and CALML5) of the five proteins identified in Example 3 were also found to be upregulated in IPF in comparison to in healthy subjects. Further, differential expression analysis was performed for transcriptomes of the lung tissues from 103 IPF and 103 HS. (Furusawa et al., “Chronic Hypersensitivity Pneumonitis, an Interstitial Lung Disease with Distinct Molecular Signatures,” Am. J. Respir. Crit. Care Med. 2020: 202(10):1430-44.) 1299 upregulated genes and 2359 downregulated genes from IPF lung tissue were identified (FIGS. 18 and 20).

TABLE 3

| Protein Symbol | FDR (BH) | Log2FC |
|---|---|---|
| PTRF/CAVIN1 | 0.003620452 | −5.666933957 |
| CAB39 | 0.001486659 | −4.381413748 |
| IQCE | 0.001486659 | −4.373319927 |
| ACAN | 0.001486659 | −4.119451163 |
| PDHA2 | 0.012723037 | −3.473594016 |
| GGH | 0.002596949 | −3.33544014 |
| COX7A2 | 0.007967339 | −3.164063015 |
| ANXA5 | 0.00907783 | −3.147676362 |
| CD47 | 0.004412021 | −3.095307394 |
| SDF4 | 0.012021033 | −3.05301955 |
| TMX2 | 0.013111663 | −2.823236916 |
| EZR | 0.008628181 | −2.752554205 |
| ALDH2 | 0.01914817 | −2.653780446 |
| LGALS3 | 0.01034415 | −2.498942007 |
| PYCARD | 0.011488902 | −2.242476365 |
| DSC3 | 0.023672189 | −2.194342238 |
| RAC2 | 0.027741621 | −2.194085271 |
| ACOX1 | 0.011488902 | −2.149811998 |
| PPIB | 0.036135955 | −2.040563177 |
| DSTN | 0.028034145 | −1.956803993 |
| TFPI | 0.017096581 | −1.938021551 |
| C12orf5/TIGAR | 0.00907783 | −1.937430867 |
| GNAI2 | 0.001486659 | −1.921477572 |
| SERPINB7 | 0.014654212 | −1.918516255 |
| MME | 0.021454533 | −1.811411479 |
| PABPC1 | 0.017096581 | −1.777446767 |
| SSR4 | 0.01744549 | −1.711124382 |
| SULT2B1 | 0.049429492 | −1.543917032 |
| LCN1 | 0.013576696 | −1.53072172 |
| PTPRZ1 | 0.039104886 | −1.522787413 |
| PRKCSH | 0.046627038 | −1.348275827 |
| ARG1 | 0.013111663 | −1.224671576 |
| TNC | 0.030529608 | 0.71548431 |
| INHBE | 0.001486659 | 0.725132102 |
| CD5L | 0.028772294 | 0.72728206 |
| PRTN3 | 0.001486659 | 0.806001118 |
| LGALS3BP | 0.014654212 | 0.830213263 |
| PODXL | 0.007967339 | 0.928297062 |
| SERPINF2 | 0.033797865 | 0.972978392 |
| HSPB1 | 0.028034145 | 0.978060655 |
| COLEC10 | 0.004412021 | 0.979694661 |
| CAMP | 0.046676379 | 0.985650991 |
| PRG4 | 0.004412021 | 1.009141731 |
| ATRN | 0.001486659 | 1.048123896 |
| DSC1 | 0.00539892 | 1.116570549 |
| PRDX2 | 0.013111663 | 1.144240558 |
| ITIH2 | 0.002596949 | 1.158366788 |
| SHBG | 0.023163109 | 1.18049439 |
| FCN1 | 0.013111663 | 1.199905377 |
| MBL2 | 0.006411218 | 1.228179912 |
| TTR | 0.035170109 | 1.257762323 |
| LBP | 0.012021033 | 1.350378468 |
| PFKL | 0.001486659 | 1.386518849 |
| TTN | 0.045467663 | 1.390696537 |
| AP1B1 | 0.01802072 | 1.405068049 |
| GGCT | 0.044042279 | 1.409217427 |
| PROL1/OPRPN | 0.011488902 | 1.43484314 |
| FETUB | 0.00907783 | 1.474320737 |
| SERF2 | 0.029661538 | 1.477267705 |
| HEG1 | 0.001486659 | 1.501683756 |
| ADIPOQ | 0.014654212 | 1.535035386 |
| FBLN1 | 0.028319489 | 1.557436188 |
| PFKP | 0.020920816 | 1.596222092 |
| ZNF511-PRAP1 | 0.026788273 | 1.627256386 |
| JUP | 0.001486659 | 1.657782473 |
| SFTPD | 0.008628181 | 1.673492131 |
| FLG2 | 0.001486659 | 1.673914277 |
| GSN | 0.00989802 | 1.67650467 |
| HAS2 | 0.001486659 | 1.727245524 |
| PCYOX1 | 0.001486659 | 1.744199863 |
| LGALS7/LGALS7B | 0.01744549 | 1.744686081 |
| MUC7 | 0.001486659 | 1.753862109 |
| CARHSP1 | 0.030098172 | 1.779325031 |
| DSP | 0.001486659 | 1.799033411 |
| SFN | 0.00907783 | 1.810123878 |
| SSC5D | 0.046627038 | 1.844957099 |
| LTF | 0.001486659 | 1.853745453 |
| RELN | 0.004412021 | 1.858939498 |
| CALML3 | 0.046970606 | 1.933699484 |
| DCDC1/DCDC5 | 0.002596949 | 1.962198005 |
| SLC4A1 | 0.007967339 | 1.975159453 |
| LAMC1 | 0.002596949 | 1.999065628 |
| CALR | 0.041144518 | 2.007967374 |
| PFN1 | 0.01034415 | 2.025607516 |
| CDSN | 0.037670432 | 2.039504612 |
| ATP5F1A | 0.039104886 | 2.048283187 |
| AMPD3 | 0.001486659 | 2.06651307 |
| GGT2 | 0.033797865 | 2.081791819 |
| PKP1 | 0.001486659 | 2.11119167 |
| CD226 | 0.046676379 | 2.119430167 |
| PTBP1 | 0.03318748 | 2.126686803 |
| LAP3 | 0.011488902 | 2.134309946 |
| TNC | 0.030529608 | 2.137049461 |
| ARHGAP1 | 0.002596949 | 2.13995453 |
| ITGA2B | 0.01914817 | 2.176526477 |
| BIN2 | 0.001486659 | 2.180326705 |
| NAMPT | 0.034782699 | 2.194424942 |
| FCN2 | 0.001486659 | 2.198053938 |
| SERPINA1 | 0.01600531 | 2.219879066 |
| SVEP1 | 0.001486659 | 2.245130212 |
| YWHAH | 0.022647419 | 2.268076851 |
| CSTA | 0.001486659 | 2.268788253 |
| SPARCL1 | 0.001486659 | 2.279597295 |
| CREG1 | 0.008628181 | 2.299255033 |
| EVPL | 0.027741621 | 2.307517025 |
| GSN | 0.00989802 | 2.316179365 |
| RAP1B | 0.001486659 | 2.316363311 |
| BLMH | 0.001486659 | 2.345994708 |
| AMY1A | 0.038611379 | 2.351644796 |
| GC | 0.012021033 | 2.387626892 |
| CCDC73 | 0.043161859 | 2.388609701 |
| CST4 | 0.033797865 | 2.393102066 |
| NCCRP1 | 0.001486659 | 2.411709151 |
| SELP | 0.02038003 | 2.418575182 |
| CALM3/CALM2/CALM1 | 0.011488902 | 2.451200005 |
| BCHE | 0.001486659 | 2.478956457 |
| SH3BGRL3 | 0.028319489 | 2.487311257 |
| MARCO | 0.001486659 | 2.492467085 |
| ACTN1 | 0.003620452 | 2.527390604 |
| CFP | 0.001486659 | 2.53133354 |
| LDHB | 0.03318748 | 2.541302887 |
| SNRPD3 | 0.001486659 | 2.582340095 |
| VTN | 0.001486659 | 2.595270266 |
| DMBT1 | 0.014654212 | 2.615476584 |
| COMP | 0.001486659 | 2.627172139 |
| MUC5B | 0.001486659 | 2.630800453 |
| GM2A | 0.01034415 | 2.650262963 |
| SFTPA1 | 0.013576696 | 2.673683105 |
| ZCCHC12 | 0.029661538 | 2.700839471 |
| CALML5 | 0.001486659 | 2.705746557 |
| YWHAE | 0.013576696 | 2.721711229 |
| CASP14 | 0.001486659 | 2.741079033 |
| IL36G | 0.003620452 | 2.773611853 |
| GAPDH | 0.002596949 | 2.815185614 |
| SELPLG | 0.047798817 | 2.822414615 |
| PTPRJ | 0.01034415 | 2.830497785 |
| CFL1 | 0.001486659 | 2.847777762 |
| FLG | 0.001486659 | 2.86687653 |
| ASAHI | 0.007327106 | 2.869175918 |
| DDX60L | 0.00907783 | 2.885247358 |
| IVL | 0.008628181 | 2.89779604 |
| CSNK1A1L | 0.001486659 | 2.976093196 |
| TNXB | 0.003620452 | 2.985342212 |
| CAPN1 | 0.004412021 | 3.000917228 |
| DHTKD1 | 0.001486659 | 3.001083751 |
| ZYX | 0.002596949 | 3.030698927 |
| STAT3 | 0.002596949 | 3.033865849 |
| PRR4 | 0.001486659 | 3.044957398 |
| SPRR3 | 0.049152669 | 3.051918908 |
| CAPG | 0.007967339 | 3.053724171 |
| CALD1 | 0.007327106 | 3.054779469 |
| CHL1 | 0.001486659 | 3.065000969 |
| CSTB | 0.00907783 | 3.093301651 |
| ALOX12B | 0.001486659 | 3.105806079 |
| ACTN4 | 0.01034415 | 3.10816556 |
| LCAT | 0.007967339 | 3.114615482 |
| CAP1 | 0.01744549 | 3.122920986 |
| C1QTNF3-AMACR | 0.001486659 | 3.155674131 |
| TMCO1 | 0.001486659 | 3.164268926 |
| TGM1 | 0.001486659 | 3.164845819 |
| TGM3 | 0.001486659 | 3.176465477 |
| HSPG2 | 0.002596949 | 3.335028031 |
| RAB15 | 0.001486659 | 3.371794877 |
| HMGB1 | 0.001486659 | 3.392365043 |
| SLC2A2 | 0.004412021 | 3.428547041 |
| HMCN1 | 0.003620452 | 3.430668097 |
| CRNN | 0.002596949 | 3.434458377 |
| ABCC2 | 0.001486659 | 3.48056511 |
| VIM | 0.001486659 | 3.508576381 |
| CARD9 | 0.003620452 | 3.649198867 |
| APMAP | 0.004412021 | 3.691991418 |
| HRNR | 0.001486659 | 3.752622398 |
| SFTPB | 0.001486659 | 3.863922068 |
| VCL | 0.001486659 | 3.939590394 |
| CKMT1A | 0.001486659 | 4.084069522 |
| PNP | 0.001486659 | 4.084367923 |
| NR0B1 | 0.001486659 | 4.150251825 |
| GPR126/ADGRG6 | 0.001486659 | 4.548325365 |
| FBN1 | 0.001486659 | 4.855105716 |
| SBSN | 0.001486659 | 5.211991792 |
| ANPEP | 0.001486659 | 5.24179039 |
| AQR | 0.001486659 | 5.387815584 |
| ANGPTL6 | 0.001486659 | 5.424441222 |
| MENT | 0.001486659 | 5.437532738 |
| FERMT3 | 0.001486659 | 6.113355725 |
| ST13P4 | 0.001486659 | 6.281156031 |
| LORICRIN | 0.001486659 | 7.251469458 |
| ALDOA | 0.001486659 | 9.172324908 |

Any of the above methods, biomarker panels or similar variants thereof can be described in various documentation associated with a product or kit. This documentation can include, without limitation, protocols, statistical analysis plans, investigator brochures, clinical guidelines, medication guides, risk evaluation and mediation programs, prescribing information and other documentation that may be associated with a pharmaceutical or diagnostic product. It is specifically contemplated that such documentation may be physically packaged with a diagnostic or pharmaceutical product according to the present disclosure as a kit, as may be beneficial or as set forth by regulatory authorities.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

We claim:

1. A method of preparing a human subject sample containing EVs and exosomes comprising:
extracting a human subject sample;
producing a sample from said human subject sample, wherein the human subject sample contains EVs and a subpopulation enriched in exosomes; and
performing proteomic analysis of plasma extracellular vesicles (EVs) in the sample containing EVs and the subpopulation enriched in exosomes.

2. The method of claim 1, further comprising quantifying levels of the at least two of High mobility group box protein 1 (HMGB1), surfactant protein B (SFTPB), Aldolase A (ALDOA), calmodulin like 5 (CALML5) and Talin-1 (TLN1) in plasma extracellular vesicles (EVs) in the sample containing EVs and the subpopulation enriched in exosomes.

3. The method of claim 1, wherein the human subject sample is blood plasma containing plasma extracellular vesicles.

4. The method of claim 1, wherein the human subject sample is not blood serum, not broncho-alveolar lavage fluid (BALF), not inflammatory cells, and/or not hyperplasic epithelial cells.

5. The method of claim 1, wherein the EVs are 50-200 nm in diameter.

6. The method of claim 1, comprising performing mass spectrometry on the sample containing EVs and the subpopulation enriched in exosomes.

7. The method of claim 2, comprising quantifying levels of the at least three of HMGB1, SFTPB, ALDOA, CALML5 and TLN1.

8. The method of claim 2, comprising quantifying levels of the at least four of HMGB1, SFTPB, ALDOA, CALML5 and TLN1.

9. A method for diagnosing idiopathic pulmonary fibrosis (IPF) using exosomal protein biomarkers, comprising:
extracting a human subject sample containing EVs and a subpopulation enriched in exosomes;
performing proteomic analysis of plasma extracellular vesicles (EVs) in the human subject sample containing EVs and the subpopulation enriched in exosomes;
identifying a protein signature for at least two exosomal protein biomarkers for IPF; and
using the protein signature to determine if the at least two exosomal protein biomarkers for IPF are elevated compared to a predetermined baseline level.

10. The method of claim 9, wherein the exosomal protein biomarkers comprise High mobility group box protein 1 (HMGB1), surfactant protein B (SFTPB), Aldolase A (ALDOA), calmodulin like 5 (CALML5) and Talin-1 (TLN1).

11. The method of claim 9, wherein a definitive diagnosis of IPF cannot be made on the sample based on high-resolution computed tomography (HRCT).

12. The method of claim 9, comprising performing the method in addition to high-resolution computed tomography (HRCT) and screening for idiopathic pulmonary fibrosis (IPF).

13. The method of claim 9, wherein the human subject sample is blood plasma containing plasma extracellular vesicles.

14. The method of claim 9, wherein the human subject sample is not blood serum, not broncho-alveolar lavage fluid (BALF), not inflammatory cells, and/or not hyperplasic epithelial cells.

15. The method of claim 9, wherein the EVs are 50-200 nm in diameter.

16. The method of claim 9, comprising performing mass spectrometry on the human sample.

17. The method of claim 9, wherein the predetermined baseline level is a level of the at least two exosomal protein biomarkers for IPF in a healthy subject.

18. The method of claim 9, wherein the predetermined baseline level is a level of the at least two exosomal protein biomarkers for IPF in a subject suffering from an ILD other than IPF.

19. The method of claim 9, further comprising differentiating idiopathic pulmonary fibrosis (IPF) from other interstitial lung diseases (ILDs) using exosomal protein biomarkers that are elevated more than a baseline specific to other ILDs other than IPF.

20. The method of claim 19, wherein the exosomal protein biomarkers comprise SFTPB, ALDOA, HMGB1, CALML5, and TLN1.

21. The method of claim 9, comprising performing the method in addition to high-resolution computed tomography (HRCT) and screening for idiopathic pulmonary fibrosis (IPF).

22. A method for screening for idiopathic pulmonary fibrosis (IPF) using exosomal protein biomarkers comprising:
preparing the sample containing EVs and the subpopulation enriched in exosomes according to claim 1;
identifying a protein signature for at least two protein biomarkers selected from the group consisting of: SFTPB, ALDOA, HMGB1, CALML5, and TLN1 in the sample containing EVs and the subpopulation enriched in exosomes; and
using the protein signature to determine if the at least two biomarkers protein biomarkers are elevated compared to a predetermined baseline level for the at least two biomarkers.

23. The method of claim 22, wherein a definitive diagnosis of IPF cannot be made based on high-resolution computed tomography (HRCT).

24. A diagnostic test kit for idiopathic pulmonary fibrosis (IPF) comprising a protein biomarker panel comprising at least two exosomal protein biomarkers for IPF and instructions for performing the method of claim 1.

25. The diagnostic test kit of claim 24, wherein the exosomal protein biomarkers are selected from the group consisting of SFTPB, ALDOA, HMGB1, CALML5, and TLN1.

26. A biomarker panel comprising at least two exosomal protein biomarkers for IPF and instructions for performing the method of claim 1.

27. The biomarker panel of claim 26, wherein the exosomal protein biomarkers are selected from the group consisting of SFTPB, ALDOA, HMGB1, CALML5, and TLN1.

28. A method for treating a subject having IPF comprising performing the method of claim 8 to diagnose the subject with IPF and administering a therapeutic for IPF to the subject.

29. The method of claim 28, wherein the therapeutic is pirfenidone, nintedanib, or a combination thereof.
